(19) United States
(12) Patent Application Publication
(10) Pub. No.: US 2026/0314600 A1
(43) Pub. Date: Oct. 15, 2026

(54) PHENYL ISOXAZOLE DERIVATIVE CONTAINING TRIAZOLE RING, PREPARATION METHOD THEREFOR, AND APPLICATION THEREOF

(71) Applicant: Guizhou University, Guiyang (CN)

(72) Inventors: Jian WU, Guiyang (CN); Bihong TIAN, Guiyang (CN); Chaokai YANG, Guiyang (CN); Jianrong YANG, Guiyang (CN); Shunhong CHEN, Guiyang (CN); Kuan TIAN, Guiyang (CN); Xinyi ZHANG, Guiyang (CN); Xiaolan YE, Guiyang (CN); Meina SONG, Guiyang (CN)

(21) Appl. No.: 19/573,591

(22) Filed: Mar. 20, 2026

(30) Foreign Application Priority Data
Mar. 26, 2025 (CN) 202510360887.3

(51) Int. Cl. A01N 43/80; A01P 7/04; C07D 413/10

(52) U.S. Cl. CPC A01N 43/80; A01P 7/04; C07D 413/10

(57) ABSTRACT

The present disclosure relates to the technical field of pesticide chemistry, and discloses a phenyl isoxazole derivative containing a triazole ring, a preparation method therefor and an application thereof. The phenyl isoxazole derivative containing a triazole ring has a structural formula of

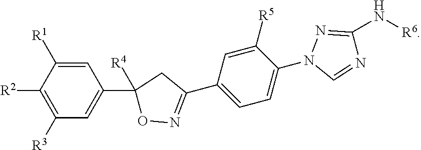

The phenyl isoxazole derivative containing a triazole ring provided by the present disclosure exhibits good insecticidal activity, at a concentration of 50 μg/mL, against agricultural pests such as Plutella xylostella, Spodoptera frugiperda, Helicoverpa armigera, Ostrinia nubilalis, and aphids. Therefore, the phenyl isoxazole derivative can be used as an active pesticide ingredient for controlling agricultural pests.

### CROSS-REFERENCE TO RELATED APPLICATIONS

The present application claims priority to Chinese Patent Application No. 202510360887.3, filed on Mar. 26, 2025, the entire disclosure of which is incorporated herein by reference.

### TECHNICAL FIELD

The present disclosure relates generally to the technical field of pesticide chemistry, and in particular, to a phenyl isoxazole derivative containing a triazole ring, a preparation method therefor, and an application thereof.

### BACKGROUND

Isoxazole compounds have been widely used in various insecticides along with the development of green pesticides. For example, isoxazole-based insecticides such as Fluralaner, Fluxametamide, Isocycloseram, Tioxazafen, and Afoxolaner all contain isoxazole moieties. Isoxazole insecticides are highly favored by agricultural professionals due to their advantages such as high activity, low toxicity, and long-lasting efficacy. They exhibit significant insecticidal activity against a wide range of pests including Lepidoptera, Hemiptera, Thysanoptera, Coleoptera, Diptera, and Acarina.

Triazole fungicides containing triazole heterocyclic derivatives have attracted great attention due to their broad-spectrum activity against pathogens of Ascomycota, Basidiomycota, and Deuteromycota. Furthermore, triazole heterocyclic derivatives also exhibit some plant growth regulating activities, and this makes it possible for use in agricultural production. Therefore, triazole fungicides are enabled to be prominent in the fungicide market.

Considering the significant insecticidal and fungicidal activities of isoxazole insecticides and triazole fungicides, the modification through conjugating an isoxazole moiety and a triazole heterocyclic moiety to synthesize a series of novel pesticides with good insecticidal activity is expected to promote the control of agricultural pests.

### SUMMARY

An object of the present disclosure is to provide a phenyl isoxazole derivative containing a triazole ring, a preparation method therefor and an application thereof. When applied as an active pesticide ingredient, the derivative exhibits excellent insecticidal activity against agricultural pests such as Plutella xylostella, Spodoptera frugiperda, Helicoverpa armigera, Ostrinia furnacalis, and aphids.

To achieve the above object, the present disclosure provides the following technical solutions:

A first aspect of the present disclosure provides a phenyl isoxazole derivative containing a triazole ring, having a structural formula of:

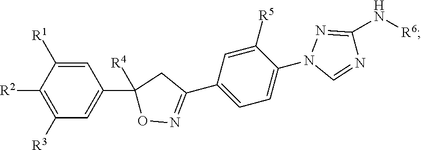

wherein, R¹, R², and R³ are each independently selected from the group consisting of hydrogen, halogen, cyano, nitro, C₁-C₆ alkyl, C₁-C₆ haloalkyl, and C₁-C₆ haloalkoxy;
R⁴ is selected from the group consisting of hydrogen, halogen, cyano, nitro, C₁-C₆ alkyl, and C₁-C₆ haloalkyl;
R⁵ is selected from the group consisting of hydrogen, halogen, cyano, nitro, hydroxyl, amino, mercapto, C₁-C₆ alkoxy, C₁-C₆ alkenyl, C₁-C₆ cycloalkyl, and aryl; and
R⁶ is selected from the group consisting of hydrogen, C₁-C₆ alkoxy, C₁-C₆ alkenyl, C₁-C₆ alkoxyoxalyl, alkanoyl, C₁-C₆ cycloalkyl, haloalkanoyl, benzoyl, substituted benzoyl, phenyl, substituted phenyl, heteroaryl, and substituted heteroaryl.

In some embodiments, R¹, R², and R³ each are hydrogen or chlorine; R⁴ is trifluoromethyl; R⁵ is cyano; R⁶ is acetyl, cycloalkanecarbonyl, methyl alkoxyoxalyl, benzoyl, or substituted benzoyl.

In some embodiments, R¹ and R³ each are chlorine; R² is hydrogen or chlorine; R⁴ is trifluoromethyl; R⁵ is cyano; R⁶ is selected from the group consisting of trifluoroacetyl, dichloroacetyl, trichloroacetyl, cycloalkanecarbonyl, methyl alkoxyoxalyl, benzoyl, 2-chloro-4-fluorobenzoyl, 3-chloro-3-chlorobenzoyl, 2-trifluoromethylbenzoyl, 3-trifluoromethylbenzoyl, 4-trifluoromethylbenzoyl, 2-fluorobenzoyl, 3-fluorobenzoyl, 4-fluorobenzoyl, 2-chlorobenzoyl, 3-chlorobenzoyl, 4-chlorobenzoyl, 2-methylbenzoyl, 3-methylbenzoyl, and 4-methylbenzoyl.

Typical structures of the phenyl isoxazole derivative containing a triazole ring according to the present disclosure include:

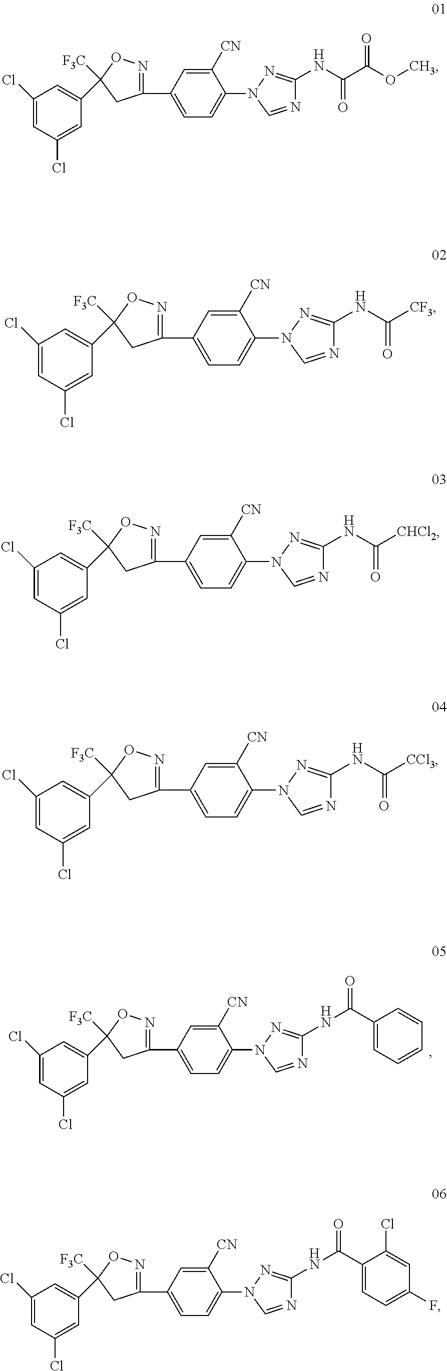

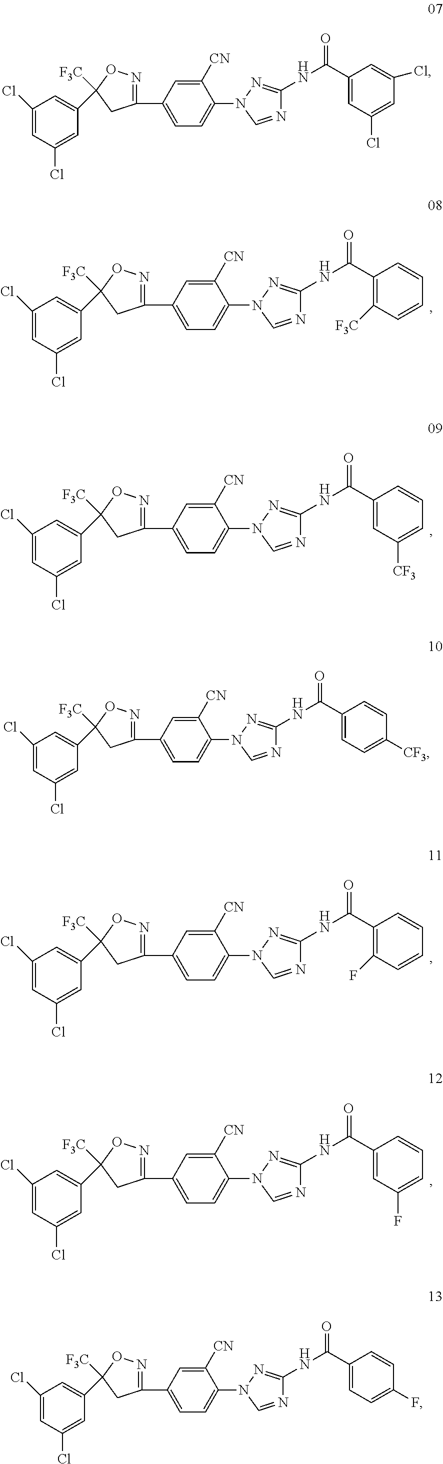

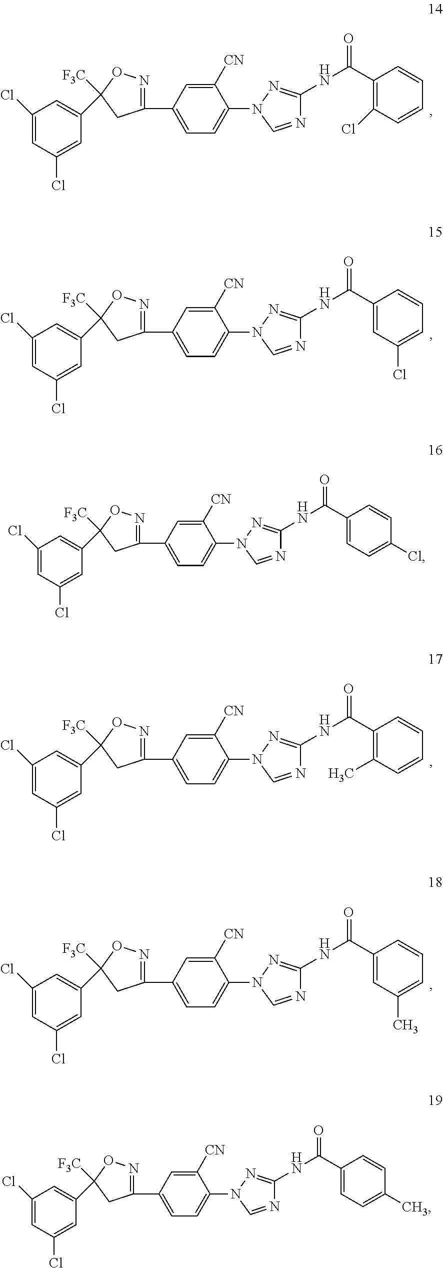 [SMILES: N#Cc1cc(C2=NOC(c3cc(Cl)cc(Cl)c3)(C(F)(F)F)C2)ccc1-n1cnc(NC(=O)c2ccccc2Cl)n1 ; N#Cc1cc(C2=NOC(c3cc(Cl)cc(Cl)c3)(C(F)(F)F)C2)ccc1-n1cnc(NC(=O)c2cccc(Cl)c2)n1 ; N#Cc1cc(C2=NOC(c3cc(Cl)cc(Cl)c3)(C(F)(F)F)C2)ccc1-n1cnc(NC(=O)c2ccc(Cl)cc2)n1 ; Cc1ccccc1C(=O)Nc1ncn(-c2ccc(C3=NOC(c4cc(Cl)cc(Cl)c4)(C(F)(F)F)C3)cc2C#N)n1 ; Cc1cccc(C(=O)Nc2ncn(-c3ccc(C4=NOC(c5cc(Cl)cc(Cl)c5)(C(F)(F)F)C4)cc3C#N)n2)c1 ; Cc1ccc(C(=O)Nc2ncn(-c3ccc(C4=NOC(c5cc(Cl)cc(Cl)c5)(C(F)(F)F)C4)cc3C#N)n2)cc1]

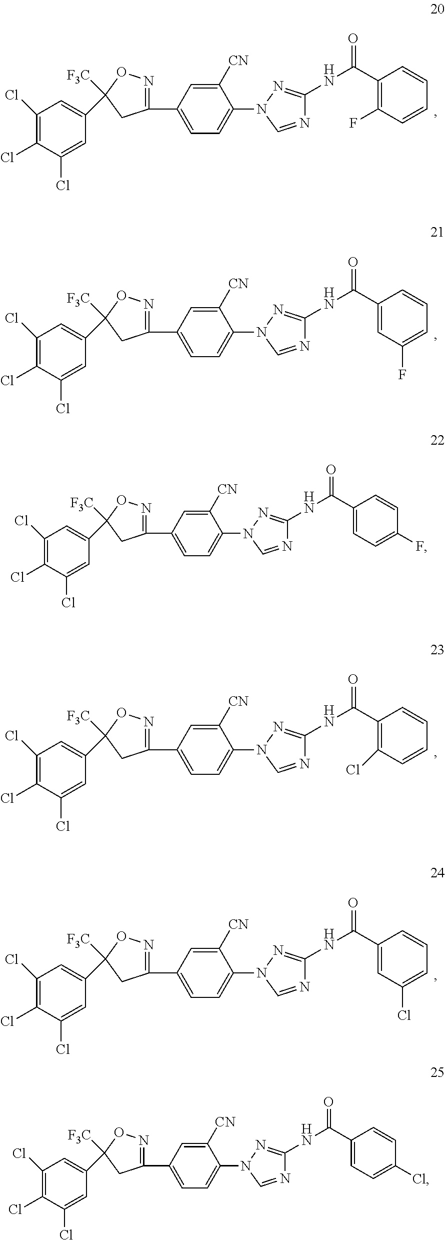 [SMILES: N#Cc1cc(C2=NOC(c3cc(Cl)c(Cl)c(Cl)c3)(C(F)(F)F)C2)ccc1-n1cnc(NC(=O)c2ccccc2F)n1 ; N#Cc1cc(C2=NOC(c3cc(Cl)c(Cl)c(Cl)c3)(C(F)(F)F)C2)ccc1-n1cnc(NC(=O)c2cccc(F)c2)n1 ; N#Cc1cc(C2=NOC(c3cc(Cl)c(Cl)c(Cl)c3)(C(F)(F)F)C2)ccc1-n1cnc(NC(=O)c2ccc(F)cc2)n1 ; N#Cc1cc(C2=NOC(c3cc(Cl)c(Cl)c(Cl)c3)(C(F)(F)F)C2)ccc1-n1cnc(NC(=O)c2ccccc2Cl)n1 ; N#Cc1cc(C2=NOC(c3cc(Cl)c(Cl)c(Cl)c3)(C(F)(F)F)C2)ccc1-n1cnc(NC(=O)c2cccc(Cl)c2)n1 ; N#Cc1cc(C2=NOC(c3cc(Cl)c(Cl)c(Cl)c3)(C(F)(F)F)C2)ccc1-n1cnc(NC(=O)c2ccc(Cl)cc2)n1]

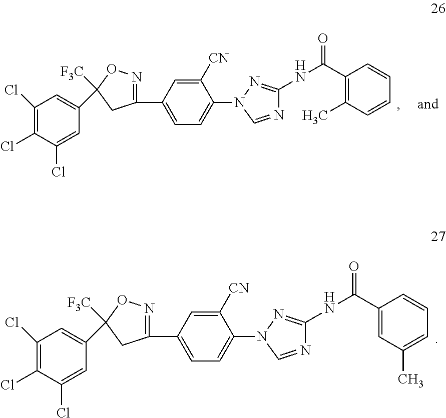 [SMILES: Cc1ccccc1C(=O)Nc1ncn(-c2ccc(C3=NOC(c4cc(Cl)c(Cl)c(Cl)c4)(C(F)(F)F)C3)cc2C#N)n1 ; Cc1cccc(C(=O)Nc2ncn(-c3ccc(C4=NOC(c5cc(Cl)c(Cl)c(Cl)c5)(C(F)(F)F)C4)cc3C#N)n2)c1]

A second aspect of the present disclosure provides a method for preparing the phenyl isoxazole derivative containing a triazole ring, comprising the following steps:
1) mixing Compound A, hydroxylamine hydrochloride, sodium carbonate, and an organic solvent for reaction to obtain Compound B, wherein Compound A has a structural formula of

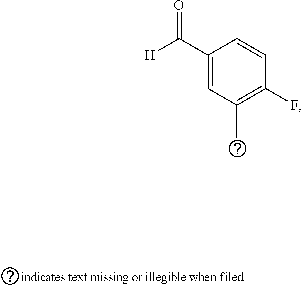

and Compound B has a structural formula of

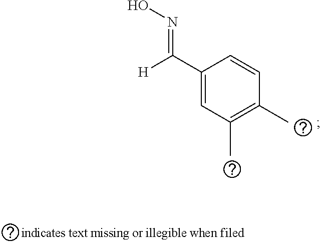

2) subjecting Compound B obtained in step 1) to a chlorination reaction to obtain a chlorinated product, and mixing the chlorinated product, Compound C, and an organic solvent for reaction to obtain Compound D, wherein Compound C has a structural formula of

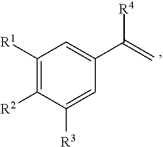

and Compound D has a structural formula of

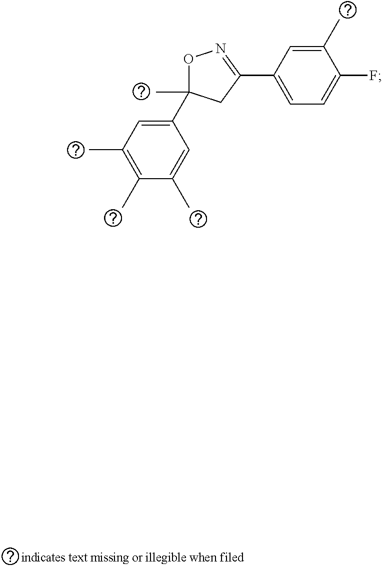 [SMILES: Cc1cc(C2=NOC(C)(c3cc(C)c(C)c(C)c3)C2)ccc1F]

3) mixing Compound D obtained in step 2), Compound E, a base, and an organic solvent for reaction to obtain Compound F, wherein Compound E has a structural formula of

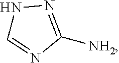

and Compound F has a structural formula of

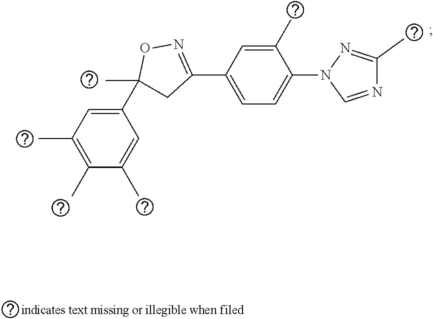

and
4) mixing Compound F obtained in step 3), R⁶, and a base for reaction to obtain the phenyl isoxazole derivative.

In some embodiments, in step 1), the organic solvent is one or more selected from the group consisting of methanol, ethanol, triethylamine, dichloromethane, chloroform, ethyl acetate, acetonitrile, toluene, xylene, tetrahydrofuran, N,N-dimethylformamide, n-propanol, and water.

In some embodiments, in step 1), a molar ratio of Compound A, hydroxylamine hydrochloride, sodium carbonate, and the organic solvent is 1:(1-3):(1-5):(2-5).

In some embodiments, in step 2), a chlorinating agent for the chlorination reaction is N-chlorosuccinimide.

In some embodiments, in step 2), the organic solvent is one or more selected from the group consisting of methanol, ethanol, triethylamine, dichloromethane, chloroform, ethyl acetate, acetonitrile, toluene, xylene, tetrahydrofuran, N,N-dimethylformamide, n-propanol, and water.

In some embodiments, in step 2), a molar ratio of Compound B, the chlorinating agent, Compound C, and the organic solvent is 1:(1-4):(1-3):(2-8).

In some embodiments, in step 3), the base is lithium hydroxide, sodium hydroxide, potassium hydroxide, calcium hydroxide, potassium tert-butoxide, aqueous ammonia, methylamine, ethylamine, propylamine, dimethylamine, diethylamine, dipropylamine, trimethylamine, triethylamine, tripropylamine, N,N-diisopropylethylamine, sodium acetate, potassium acetate, or sodium bicarbonate.

In some embodiments, in step 3), the organic solvent is one or more selected from the group consisting of methanol, ethanol, triethylamine, dichloromethane, chloroform, ethyl acetate, acetonitrile, toluene, xylene, tetrahydrofuran, N,N-dimethylformamide, n-propanol, and water.

In some embodiments, in step 3), a molar ratio of Compound D, Compound E, the base, and the organic solvent is 1:(1-4):(1-3):(2-8).

In some embodiments, in step 3), Compound E can also be

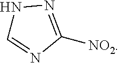

In some embodiments, when Compound E is

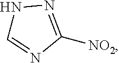

after the reaction of Compound D, the base, and the organic solvent is completed, a reducing agent is added to reduce —NO₂ to —NH₂.

In some embodiments, the reducing agent is one selected from the group consisting of Na, K, Mg, Al, Zn, Fe, Cu, LiAlH₄, NaBH₄, H₂S, Na₂SO₃ H₂C₂O₄, Na₂S₂O₄, and N₂H₄; and a molar ratio of Compound D, the reducing agent, Compound E, the base, and the organic solvent is 1:(1-4):(1-4):(1-3):(2-8).

In some embodiments, in step 4), the base is lithium hydroxide, sodium hydroxide, potassium hydroxide, calcium hydroxide, potassium tert-butoxide, aqueous ammonia, methylamine, ethylamine, propylamine, dimethylamine, diethylamine, dipropylamine, trimethylamine, triethylamine, tripropylamine, N,N-diisopropylethylamine, sodium acetate, potassium acetate, or sodium bicarbonate.

In some embodiments, in step 4), a molar ratio of Compound F, R⁶, and the base is 1:(1-4):(1-3).

The method for preparing the phenyl isoxazole derivative containing a triazole ring according to the present disclosure involves synthetic routes as shown below:

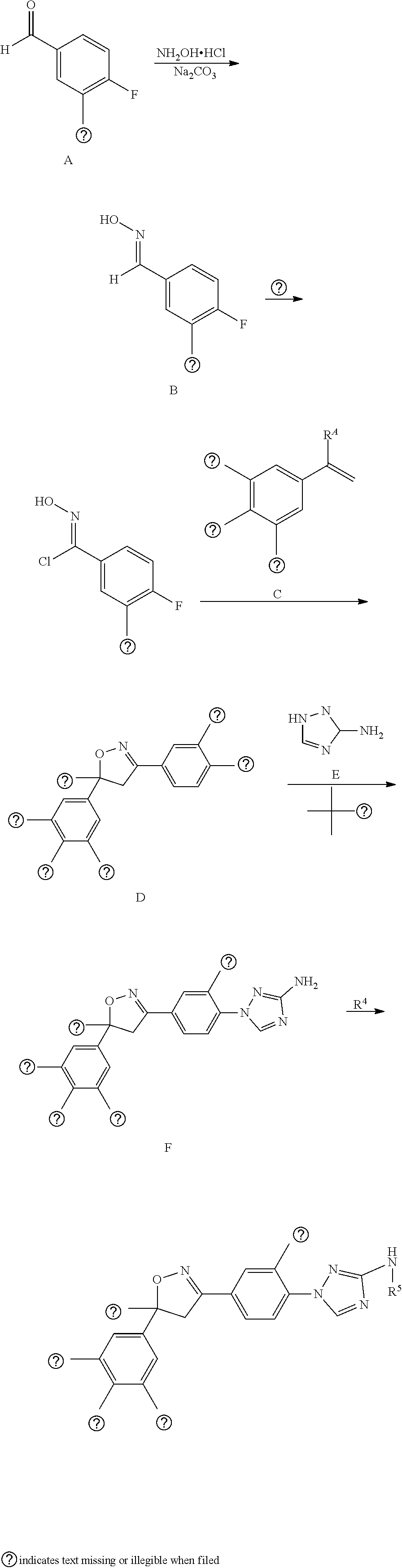

A third aspect of the present disclosure is to provide an application of the phenyl isoxazole derivative containing a triazole ring as an active pesticide ingredient in the control of agricultural pests.

In some embodiments, the agricultural pests include Plutella xylostella, Spodoptera frugiperda, Helicoverpa armigera, Ostrinia nubilalis, and aphids.

Compared with the prior art, the beneficial effects of the present disclosure are as follows:

The phenyl isoxazole derivative containing a triazole ring provided by the present disclosure exhibits good insecticidal activity, at a concentration of 50 μg/mL, against agricultural pests such as Plutella xylostella, Spodoptera frugiperda, Helicoverpa armigera, Ostrinia nubilalis, and aphids. Therefore, the phenyl isoxazole derivative of the present disclosure can be used as an active pesticide ingredient for controlling agricultural pests.

### DETAILED DESCRIPTION OF THE EMBODIMENTS

Various exemplary embodiments of the present disclosure are now described in detail. This detailed description should not be construed as limiting the invention, but rather as a more detailed explanation of certain aspects, features, and embodiments of the invention. It should be understood that the terms used herein are merely for describing particular embodiments and are not intended to limit the invention.

Additionally, for numerical ranges described herein, each intervening value between the upper and lower limits of that range is also specifically disclosed. Any smaller range between any stated value or intervening value in a stated range and any other stated value or intervening value in that stated range is encompassed within the invention. The upper and lower limits of these smaller ranges may independently be included or excluded in the range.

Unless otherwise defined, all technical and scientific terms used herein have the same meaning as commonly understood by one of ordinary skill in the art to which this invention belongs. Although only preferred methods and materials are described herein, any methods and materials similar or equivalent to those described herein can also be used in the practice or testing of the present disclosure. All documents mentioned in this specification are incorporated by reference to disclose and describe the methods and/or materials associated with the cited documents. In case of conflict with any incorporated document, the content of this specification shall control.

Various modifications and variations can be made to the specific embodiments of the present specification without departing from the scope or spirit of the invention, which will be apparent to those skilled in the art. Other embodiments obtained from the description of the present disclosure will be apparent to those skilled in the art. The description and examples of the present disclosure are merely exemplary.

The terms “comprising,” “including,” “having,” “containing,” etc. used herein are open-ended terms, meaning “including but not limited to.”

### Example 1

### Preparation of a Phenyl Isoxazole Derivative Containing a Triazole Ring

1) 3-Cyano-4-fluorobenzaldehyde (Compound A, 20.0 g, 0.13 mol), hydroxylamine hydrochloride (14 g, 0.2 mol), and a methanol-water solution (250 mL, 1:2 mol/mol) were added to a 250 mL round-bottom flask. The mixture was stirred at room temperature for 2 h. Subsequently, sodium carbonate (25 g, 0.23 mol) was slowly added, and the mixture was further stirred at room temperature for 2 h. After the reaction was completed, ice cubes were added to precipitate a solid. The solid was collected by suction filtration, dried, and purified by column chromatography to obtain (E)-2-fluoro-5-(hydroxyimino)methylbenzonitrile (Compound B) as a yellow-green solid.

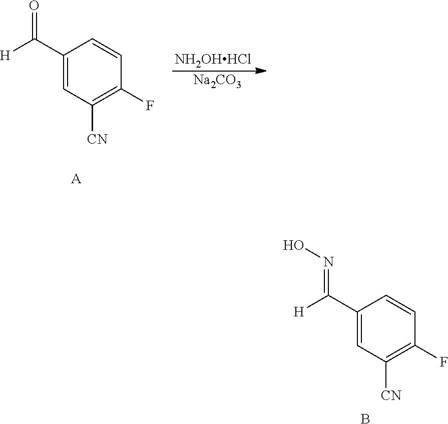

The yield of Compound B was determined to be 82%.

2) (E)-2-Fluoro-5-(hydroxyimino)methylbenzonitrile (Compound B, 16.4 g, 0.11 mol) obtained in step 1) and NCS (30 g, 0.22 mol) were added to a 250 mL round-bottom flask. The mixture was stirred at room temperature for 30 min. Subsequently, 1,3-dichloro-5-(3,3,3-trifluoroprop-1-en-2-yl)benzene (Compound C, 28.8 g, 0.12 mol) and triethylamine (13.3 g, 0.13 mol) were added. The mixture was stirred at room temperature for 18 h. After the reaction was completed, the mixture was washed with water, extracted with dichloromethane, dried over anhydrous sodium sulfate, concentrated under vacuum, and purified by column chromatography to obtain 5-(5-(3,5-dichlorophenyl)-5-(trifluoromethyl)-4,5-dihydroisoxazol-3-yl)-2-fluorobenzonitrile (Compound D) as a white solid.

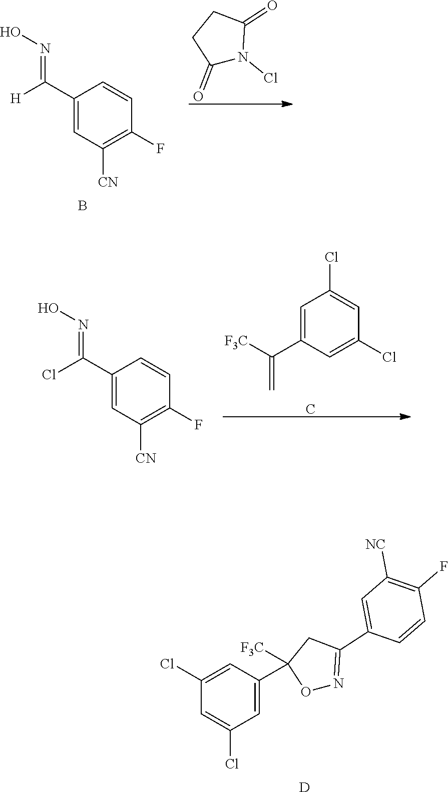

The yield of Compound D was determined to be 95.7%.

3) 3-Amino-1H-1,2,4-triazole (Compound E, 1.7 g, 20 mmol), potassium tert-butoxide (2.8 g, 2.5 mmol), and DMF (50 mL) were added to a 100 mL round-bottom flask. The mixture was stirred at room temperature for 1 h. Subsequently, 5-(5-(3,5-dichlorophenyl)-5-(trifluoromethyl)-4,5-dihydroisoxazol-3-yl)-2-fluorobenzonitrile (Compound D, 4 g, 10 mmol) obtained in step 2) was slowly added. The mixture was stirred and heated under reflux at 100° C. for 4 h. After the reaction was completed, the mixture was concentrated under vacuum and purified by column chromatography to obtain 2-(5-amino-1H-1,2,4-triazol-1-yl)-5-(5-(3,5-dichlorophenyl)-5-(trifluoromethyl)-4,5-dihydroisoxazol-3-yl)benzonitrile (Compound F) as a light yellow solid.

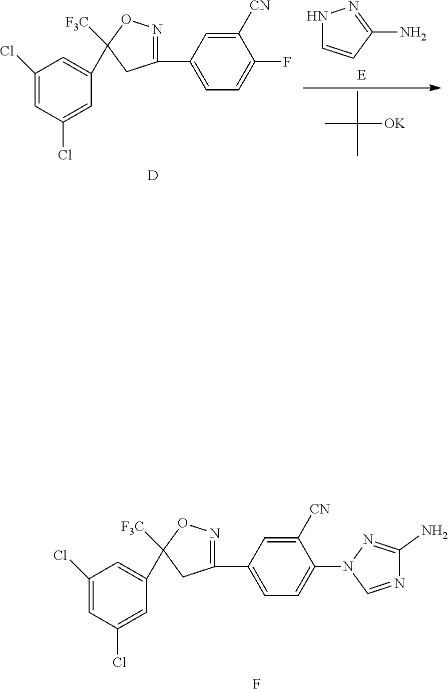

The yield of Compound F was determined to be 44%.

4) 2-(5-Amino-1H-1,2,4-triazol-1-yl)-5-(5-(3,5-dichlorophenyl)-5-(trifluoromethyl)-4,5-dihydroisoxazol-3-yl)benzonitrile (Compound F, 0.3 g, 0.64 mmol) obtained in step 3), methyl 2-chloro-2-oxoacetate (0.08 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain methyl 2-((1-(2-cyano-4-(5-(3,5-dichlorophenyl)-5-(trifluoromethyl)-4,5-dihydroisoxazol-3-yl)phenyl)-1H-1,2,4-triazol-5-yl)amino)-2-oxoacetate as a light yellow solid, which was the phenyl isoxazole derivative containing a triazole of the present disclosure.

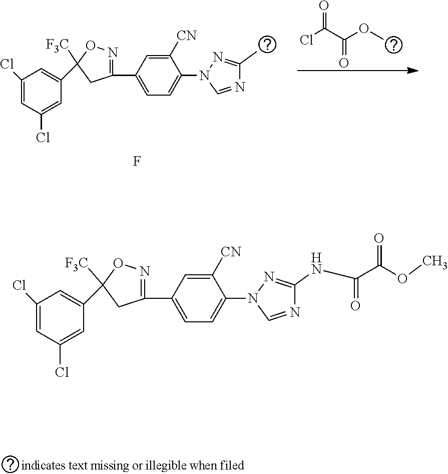

The yield of the phenyl isoxazole derivative in this example was determined to be 16%.

¹H NMR (500 MHz) δ 9.88 (s, 1H), 8.64 (s, 1H), 7.93 (s, 1H), 7.92-7.88 (m, 2H), 7.46 (s, 2H), 7.41 (d, J=0.9 Hz, 1H), 4.75 (d, J=13.8 Hz, 1H), 4.66 (s, 3H), 4.46 (d, J=13.8 Hz, 1H).

### Example 2

### Preparation of a Phenyl Isoxazole Derivative Containing a Triazole Ring

Steps 1) to 3) were the same as in Example 1.

4) 2-(5-Amino-1H-1,2,4-triazol-1-yl)-5-(5-(3,5-dichlorophenyl)-5-(trifluoromethyl)-4,5-dihydroisoxazol-3-yl)benzonitrile (Compound F, 0.3 g, 0.64 mmol) obtained in step 3), trifluoroacetyl chloride (0.12 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,5-dichlorophenyl)-5-(trifluoromethyl)-4,5-dihydroisoxazol-3-yl)phenyl)-1H-1,2,4-triazol-5-yl)-2,2,2-trifluoroacetamide as a light brown solid, which was the phenyl isoxazole derivative containing a triazole ring of the present disclosure.

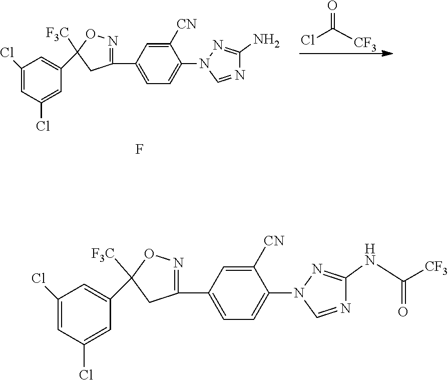

The yield of the phenyl isoxazole derivative in this example was determined to be 13.3%.

¹H NMR (600 MHz) δ 8.35 (s, 1H), 8.08 (s, 1H), 7.47 (d, J=1.4 Hz, 1H), 7.45 (dd, J=6.9, 1.4 Hz, 1H), 7.43 (d, J=6.9 Hz, 1H), 7.00 (s, 2H), 6.96-6.95 (m, 1H), 4.28 (d, J=13.7 Hz, 1H), 3.99 (d, J=13.7 Hz, 1H).

### Example 3

### Preparation of a Phenyl Isoxazole Derivative Containing a Triazole Ring

Steps 1) to 3) were the same as in Example 1.

4) 2-(5-Amino-1H-1,2,4-triazol-1-yl)-5-(5-(3,5-dichlorophenyl)-5-(trifluoromethyl)-4,5-dihydroisoxazol-3-yl)benzonitrile (Compound F, 0.3 g, 0.64 mmol) obtained in step 3), dichloroacetyl chloride (0.1 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,5-dichlorophenyl)-5-(trifluoromethyl)-4,5-dihydroisoxazol-3-yl)phenyl)-1H-1,2,4-triazol-5-yl)-2,2-dichloroacetamide as a brown solid, which was the phenyl isoxazole derivative containing a triazole ring of the present disclosure.

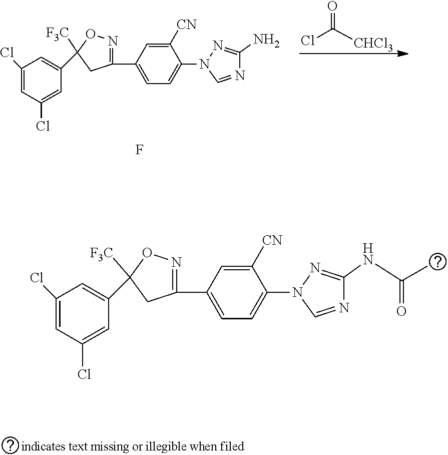

The yield of the phenyl isoxazole derivative in this example was determined to be 12.5%.

¹H NMR (500 MHz) δ 8.52 (s, 1H), 8.27 (s, 1H), 7.68-7.63 (m, 2H), 7.61 (d, J=6.9 Hz, 1H), 7.19 (s, 2H), 7.16 (t, J=1.3 Hz, 1H), 6.08 (s, 1H), 4.48 (d, J=13.8 Hz, 1H), 4.19 (d, J=13.8 Hz, 1H).

### Example 4

### Preparation of a Phenyl Isoxazole Derivative Containing a Triazole Ring

Steps 1) to 3) were the same as in Example 1.

4) 2-(5-Amino-1H-1,2,4-triazol-1-yl)-5-(5-(3,5-dichlorophenyl)-5-(trifluoromethyl)-4,5-dihydroisoxazol-3-yl)benzonitrile (Compound F, 0.3 g, 0.64 mmol) obtained in step 3), trichloroacetyl chloride (0.1 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,5-dichlorophenyl)-5-(trifluoromethyl)-4,5-dihydroisoxazol-3-yl)phenyl)-1H-1,2,4-triazol-5-yl)-2,2,2-trichloroacetamide as a white solid, which was the phenyl isoxazole derivative containing a triazole ring of the present disclosure.

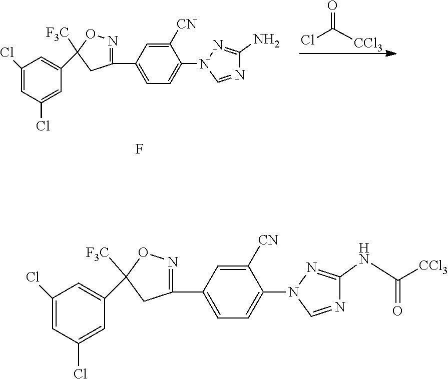

The yield of the phenyl isoxazole derivative in this example was determined to be 41.7%.

¹H NMR (500 MHz) δ 8.55 (s, 1H), 8.29 (s, 1H), 7.67 (s, 1H), 7.66-7.61 (m, 2H), 7.20 (s, 2H), 7.16 (d, J=1.3 Hz, 1H), 4.49 (d, J=13.8 Hz, 1H), 4.19 (d, J=13.8 Hz, 1H).

### Example 5

### Preparation of a Phenyl Isoxazole Derivative Containing a Triazole Ring

Steps 1) to 3) were the same as in Example 1.

4) 2-(5-Amino-1H-1,2,4-triazol-1-yl)-5-(5-(3,5-dichlorophenyl)-5-(trifluoromethyl)-4,5-dihydroisoxazol-3-yl)benzonitrile (Compound F, 0.3 g, 0.64 mmol) obtained in step 3), benzoyl chloride (0.09 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,5-dichlorophenyl)-5-(trifluoromethyl)-4,5-dihydroisoxazol-3-yl)phenyl)-1H-1,2,4-triazol-5-yl)benzamide as a light yellow solid, which was the phenyl isoxazole derivative containing a triazole ring of the present disclosure.

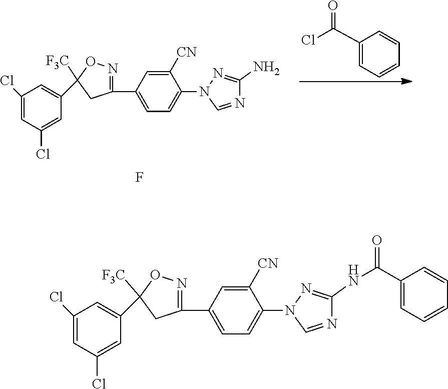

The yield of the phenyl isoxazole derivative in this example was determined to be 50%.

¹H NMR (500 MHz) δ 9.40 (s, 1H), 8.04 (s, 1H), 7.64 (s, 1H), 7.55 (dd, J=10.5, 4.7 Hz, 4H), 7.24 (t, J=5.9 Hz, 1H), 7.20-7.17 (m, 4H), 7.12 (t, J=1.5 Hz, 1H), 4.49 (d, J=13.8 Hz, 1H), 4.22 (d, J=13.9 Hz, 1H).

### Example 6

### Preparation of a Phenyl Isoxazole Derivative Containing a Triazole Ring

Steps 1) to 3) were the same as in Example 1.

4) 2-(5-Amino-1H-1,2,4-triazol-1-yl)-5-(5-(3,5-dichlorophenyl)-5-(trifluoromethyl)-4,5-dihydroisoxazol-3-yl)benzonitrile (Compound F, 0.3 g, 0.64 mmol) obtained in step 3), 2-chloro-4-fluorobenzoyl chloride (0.12 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,5-dichlorophenyl)-5-(trifluoromethyl)-4,5-dihydroisoxazol-3-yl)phenyl)-1H-1,2,4-triazol-5-yl)-2-chloro-4-fluorobenzamide as a light yellow solid, which was the phenyl isoxazole derivative containing a triazole ring of the present disclosure.

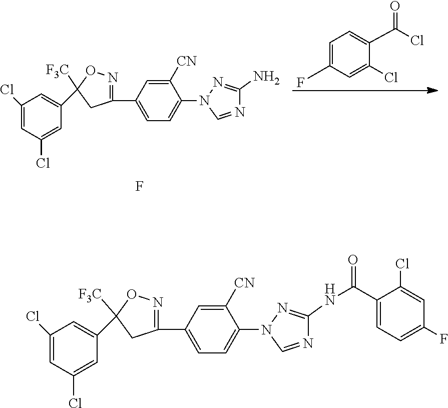

The yield of the phenyl isoxazole derivative in this example was determined to be 50%.

¹H NMR (500 MHz) δ 9.76 (s, 1H), 7.97 (s, 1H), 7.67 (s, 1H), 7.62-7.57 (m, 2H), 7.40-7.34 (m, 1H), 7.17 (s, 2H), 7.12 (d, J=0.7 Hz, 1H), 6.97-6.92 (m, 1H), 6.88 (dd, J=9.2, 3.8 Hz, 1H), 4.49 (d, J=13.9 Hz, 1H), 4.24 (d, J=13.9 Hz, 1H).

### Example 7

### Preparation of a Phenyl Isoxazole Derivative Containing a Triazole Ring

Steps 1) to 3) were the same as in Example 1.

4) 2-(5-Amino-1H-1,2,4-triazol-1-yl)-5-(5-(3,5-dichlorophenyl)-5-(trifluoromethyl)-4,5-dihydroisoxazol-3-yl)benzonitrile (Compound F, 0.3 g, 0.64 mmol) obtained in step 3), 3,5-dichlorobenzoyl chloride (0.13 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,5-dichlorophenyl)-5-(trifluoromethyl)-4,5-dihydroisoxazol-3-yl)phenyl)-1H-1,2,4-triazol-5-yl)-3,5-dichlorobenzamide as a light yellow solid, which was the phenyl isoxazole derivative containing a triazole ring of the present disclosure.

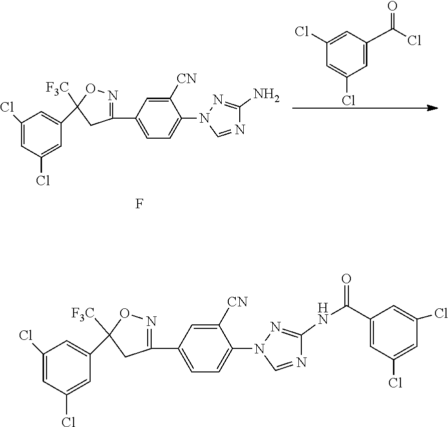

The yield of the phenyl isoxazole derivative in this example was determined to be 33.3%.

¹H NMR (600 MHz) δ 9.08 (s, 1H), 8.80 (s, 1H), 8.10-8.08 (m, 1H), 8.07-8.03 (m, 2H), 7.83 (d, J=1.9 Hz, 2H), 7.58 (t, J=1.8 Hz, 1H), 7.51 (d, J=1.6 Hz, 2H), 7.46 (dd, J=2.4, 1.2 Hz, 1H), 4.12 (d, J=17.1 Hz, 1H), 3.75 (d, J=17.2 Hz, 1H).

### Example 8

### Preparation of a Phenyl Isoxazole Derivative Containing a Triazole Ring

Steps 1) to 3) were the same as in Example 1.

4) 2-(5-Amino-1H-1,2,4-triazol-1-yl)-5-(5-(3,5-dichlorophenyl)-5-(trifluoromethyl)-4,5-dihydroisoxazol-3-yl)benzonitrile (Compound F, 0.3 g, 0.64 mmol) obtained in step 3), 2-(trifluoromethyl)benzoyl chloride (0.13 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,5-dichlorophenyl)-5-(trifluoromethyl)-4,5-dihydroisoxazol-3-yl)phenyl)-1H-1,2,4-triazol-5-yl)-2-(trifluoromethyl)benzamide as a light yellow solid, which was the phenyl isoxazole derivative containing a triazole ring of the present disclosure.

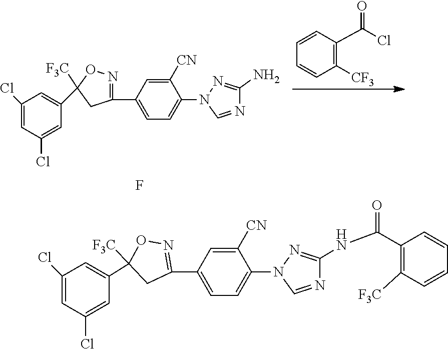

The yield of the phenyl isoxazole derivative in this example was determined to be 26.7%.

¹H NMR (500 MHz) δ 9.83 (s, 1H), 7.67 (s, 1H), 7.58 (t, J=18.9 Hz, 3H), 7.43 (d, J=6.1 Hz, 1H), 7.40-7.30 (m, 3H), 7.18 (s, 2H), 7.13 (s, 1H), 4.49 (d, J=13.8 Hz, 1H), 4.22 (d, J=13.8 Hz, 1H).

### Example 9

### Preparation of a Phenyl Isoxazole Derivative Containing a Triazole Ring

Steps 1) to 3) were the same as in Example 1.

4) 2-(5-Amino-1H-1,2,4-triazol-1-yl)-5-(5-(3,5-dichlorophenyl)-5-(trifluoromethyl)-4,5-dihydroisoxazol-3-yl)benzonitrile (Compound F, 0.3 g, 0.64 mmol) obtained in step 3), 3-(trifluoromethyl)benzoyl chloride (0.13 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,5-dichlorophenyl)-5-(trifluoromethyl)-4,5-dihydroisoxazol-3-yl)phenyl)-1H-1,2,4-triazol-5-yl)-3-(trifluoromethyl)benzamide as a light yellow solid, which was the phenyl isoxazole derivative containing a triazole ring of the present disclosure.

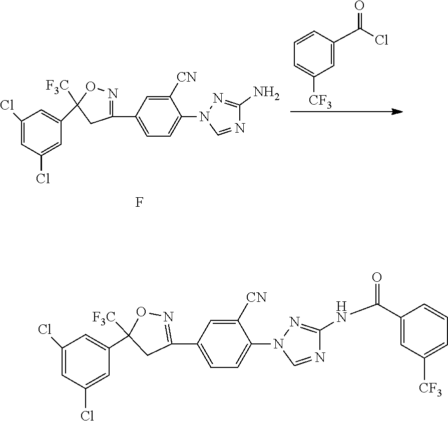

The yield of the phenyl isoxazole derivative in this example was determined to be 76.7%.

¹H NMR (600 MHz) δ 9.50 (s, 1H), 8.84 (s, 1H), 8.29 (s, 1H), 8.21 (d, J=7.8 Hz, 1H), 8.11-8.08 (m, 2H), 8.06 (dd, J=8.7, 1.9 Hz, 1H), 7.87 (d, J=7.8 Hz, 1H), 7.69 (t, J=7.8 Hz, 1H), 7.51 (d, J=1.6 Hz, 2H), 7.46 (t, J=1.8 Hz, 1H), 4.12 (d, J=17.1 Hz, 1H), 3.75 (d, J=17.2 Hz, 1H).

### Example 10

### Preparation of a Phenyl Isoxazole Derivative Containing a Triazole Ring

Steps 1) to 3) were the same as in Example 1.

4) 2-(5-Amino-1H-1,2,4-triazol-1-yl)-5-(5-(3,5-dichlorophenyl)-5-(trifluoromethyl)-4,5-dihydroisoxazol-3-yl)benzonitrile (Compound F, 0.3 g, 0.64 mmol) obtained in step 3), 4-(trifluoromethyl)benzoyl chloride (0.13 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,5-dichlorophenyl)-5-(trifluoromethyl)-4,5-dihydroisoxazol-3-yl)phenyl)-1H-1,2,4-triazol-5-yl)-4-(trifluoromethyl)benzamide as a white solid, which was the phenyl isoxazole derivative containing a triazole ring of the present disclosure.

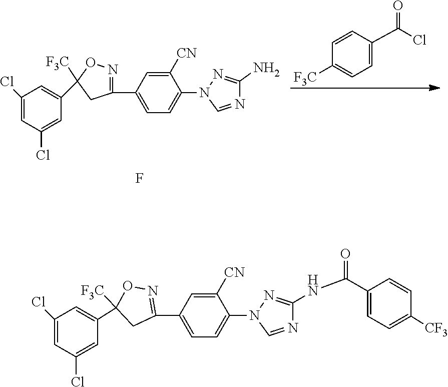

The yield of the phenyl isoxazole derivative in this example was determined to be 53.3%.

¹H NMR (600 MHz) δ 8.98 (s, 1H), 8.81 (s, 1H), 8.09 (d, J=0.9 Hz, 2H), 8.08 (td, J=4.4, 1.1 Hz, 3H), 7.80 (d, J=8.3 Hz, 2H), 7.51 (d, J=1.6 Hz, 2H), 7.46 (t, J=1.8 Hz, 1H), 4.12 (d, J=17.2 Hz, 1H), 3.75 (d, J=17.2 Hz, 1H).

### Example 11

### Preparation of a Phenyl Isoxazole Derivative Containing a Triazole Ring

Steps 1) to 3) were the same as in Example 1.

4) 2-(5-Amino-1H-1,2,4-triazol-1-yl)-5-(5-(3,5-dichlorophenyl)-5-(trifluoromethyl)-4,5-dihydroisoxazol-3-yl)benzonitrile (Compound F, 0.3 g, 0.64 mmol) obtained in step 3), 2-fluorobenzoyl chloride (0.1 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,5-dichlorophenyl)-5-(trifluoromethyl)-4,5-dihydroisoxazol-3-yl)phenyl)-1H-1,2,4-triazol-5-yl)-2-fluorobenzamide as a light yellow solid, which was the phenyl isoxazole derivative containing a triazole ring of the present disclosure.

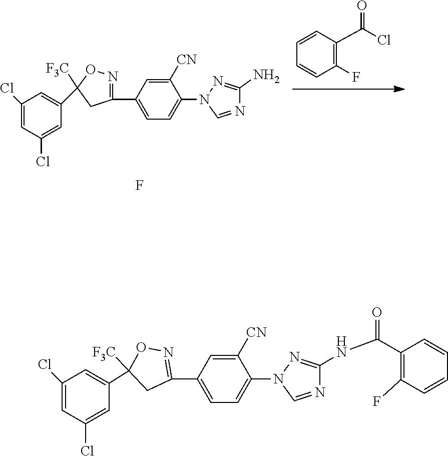

The yield of the phenyl isoxazole derivative in this example was determined to be 72.5%.

¹H NMR (400 MHz, CDCl₃) δ 9.31 (s, 2H), 9.31 (s, 1H), 8.87 (s, 2H), 8.87 (s, 2H), 8.22 (t, J=7.4 Hz, 2H), 8.11 (d, J=8.9 Hz, 4H), 8.08-8.03 (m, 2H), 7.61-7.55 (m, 2H), 7.52 (s, 3H), 7.46 (s, 2H), 7.35 (t, J=7.5 Hz, 2H), 7.27 (s, 2H), 4.13 (d, J=17.2 Hz, 2H), 4.13 (d, J=17.2 Hz, 2H), 3.76 (d, J=17.3 Hz, 2H), 3.76 (d, J=17.3 Hz, 2H).

### Example 12

### Preparation of a Phenyl Isoxazole Derivative Containing a Triazole Ring

Steps 1) to 3) were the same as in Example 1.

4) 2-(5-Amino-1H-1,2,4-triazol-1-yl)-5-(5-(3,5-dichlorophenyl)-5-(trifluoromethyl)-4,5-dihydroisoxazol-3-yl)benzonitrile (Compound F, 0.3 g, 0.64 mmol) obtained in step 3), 3-fluorobenzoyl chloride (0.1 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,5-dichlorophenyl)-5-(trifluoromethyl)-4,5-dihydroisoxazol-3-yl)phenyl)-1H-1,2,4-triazol-5-yl)-3-fluorobenzamide as a light yellow solid, which was the phenyl isoxazole derivative containing a triazole ring of the present disclosure.

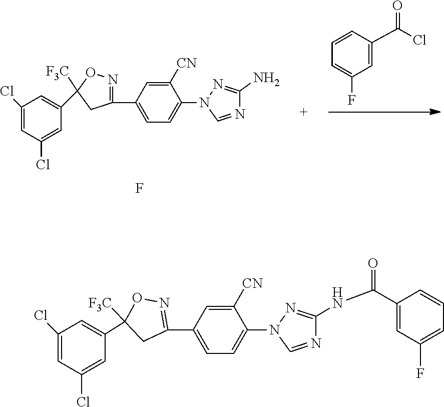

The yield of the phenyl isoxazole derivative in this example was determined to be 33.3%.

¹H NMR (400 MHz, CDCl₃) δ 10.44 (s, 1H), 8.90 (s, 1H), 8.11 (d, J=7.8 Hz, 2H), 8.04 (d, J=8.6 Hz, 1H), 7.89 (t, J=8.8 Hz, 2H), 7.80 (dd, J=18.3, 9.2 Hz, 2H), 7.52 (d, J=5.4 Hz, 2H), 7.45 (s, 1H), 4.13 (d, J=17.3 Hz, 1H), 3.77 (d, J=17.3 Hz, 1H).

### Example 13

### Preparation of a Phenyl Isoxazole Derivative Containing a Triazole Ring

Steps 1) to 3) were the same as in Example 1.

4) 2-(5-Amino-1H-1,2,4-triazol-1-yl)-5-(5-(3,5-dichlorophenyl)-5-(trifluoromethyl)-4,5-dihydroisoxazol-3-yl)benzonitrile (Compound F, 0.3 g, 0.64 mmol) obtained in step 3), 4-fluorobenzoyl chloride (0.1 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,5-dichlorophenyl)-5-(trifluoromethyl)-4,5-dihydroisoxazol-3-yl)phenyl)-1H-1,2,4-triazol-5-yl)-4-fluorobenzamide as a light yellow solid, which was the phenyl isoxazole derivative containing a triazole ring of the present disclosure.

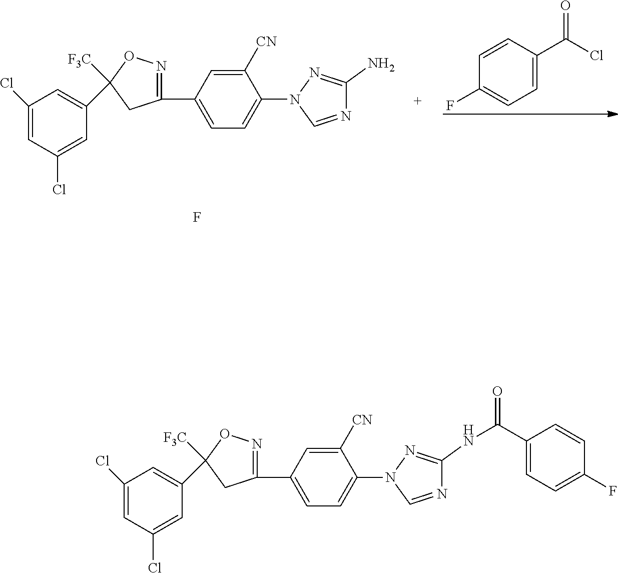

The yield of the phenyl isoxazole derivative in this example was determined to be 45.5%.

¹H NMR (400 MHz, CDCl₃) δ 10.46 (s, 1H), 8.91 (s, 1H), 8.13 (d, J=8.4 Hz, 3H), 8.05 (d, J=9.6 Hz, 1H), 7.51 (s, 1H), 7.47-7.43 (m, 1H), 7.21 (t, J=8.5 Hz, 1H), 7.18-7.08 (m, 3H), 4.13 (d, J=17.3 Hz, 1H), 3.77 (d, J=17.2 Hz, 1H).

### Example 14

### Preparation of a Phenyl Isoxazole Derivative Containing a Triazole Ring

Steps 1) to 3) were the same as in Example 1.

4) 2-(5-Amino-1H-1,2,4-triazol-1-yl)-5-(5-(3,5-dichlorophenyl)-5-(trifluoromethyl)-4,5-dihydroisoxazol-3-yl)benzonitrile (Compound F, 0.3 g, 0.64 mmol) obtained in step 3), 2-chlorobenzoyl chloride (0.11 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,5-dichlorophenyl)-5-(trifluoromethyl)-4,5-dihydroisoxazol-3-yl)phenyl)-1H-1,2,4-triazol-5-yl)-2-chlorobenzamide as a light yellow solid, which was the phenyl isoxazole derivative containing a triazole ring of the present disclosure.

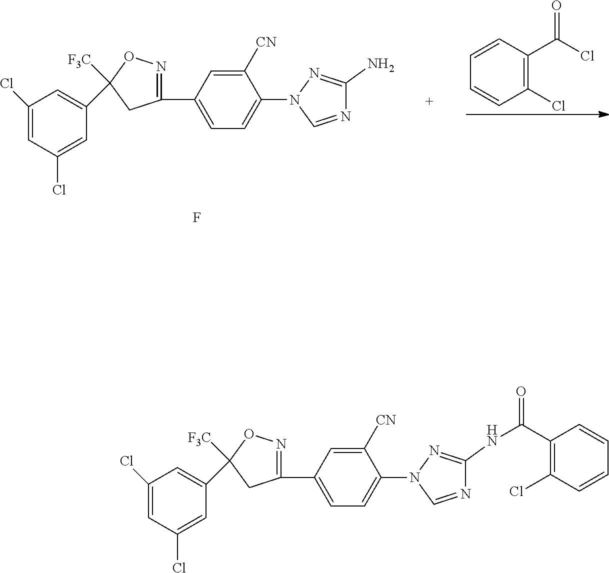

The yield of the phenyl isoxazole derivative in this example was determined to be 62%.

¹H NMR (600 MHz) δ 9.31 (s, 1H), 7.64 (s, 1H), 7.46 (s, 1H), 7.40 (dd, J=6.9, 1.3 Hz, 1H), 7.17 (d, J=5.8 Hz, 1H), 6.99 (s, 2H), 6.98-6.95 (m, 2H), 6.93 (ddd, J=6.8, 4.4, 1.3 Hz, 2H), 4.29 (d, J=13.8 Hz, 1H), 4.01 (d, J=13.8 Hz, 1H).

### Example 15

### Preparation of a Phenyl Isoxazole Derivative Containing a Triazole Ring

Steps 1) to 3) were the same as in Example 1.

4) 2-(5-Amino-1H-1,2,4-triazol-1-yl)-5-(5-(3,5-dichlorophenyl)-5-(trifluoromethyl)-4,5-dihydroisoxazol-3-yl)benzonitrile (Compound F, 0.3 g, 0.64 mmol) obtained in step 3), 3-chlorobenzoyl chloride (0.11 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,5-dichlorophenyl)-5-(trifluoromethyl)-4,5-dihydroisoxazol-3-yl)phenyl)-1H-1,2,4-triazol-5-yl)-3-chlorobenzamide as a light yellow solid, which was the phenyl isoxazole derivative containing a triazole ring of the present disclosure.

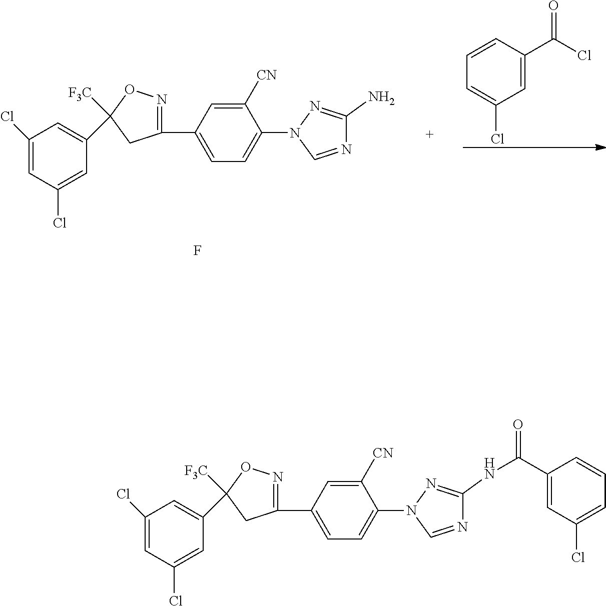

The yield of the phenyl isoxazole derivative in this example was determined to be 47.5%.

¹H NMR (600 MHz) δ 9.90 (s, 1H), 8.57 (s, 1H), 7.95 (t, J=1.2 Hz, 1H), 7.93 (d, J=1.7 Hz, 1H), 7.92 (d, J=2.6 Hz, 1H), 7.87 (dd, J=7.0, 1.5 Hz, 1H), 7.84 (t, J=6.9 Hz, 2H), 7.51-7.50 (m, 1H), 7.45 (t, J=4.9 Hz, 2H), 7.43-7.41 (m, 1H), 7.39 (t, J=6.3 Hz, 1H), 4.75 (d, J=13.7 Hz, 1H), 4.46 (d, J=13.7 Hz, 1H).

### Example 16

### Preparation of a Phenyl Isoxazole Derivative Containing a Triazole Ring

Steps 1) to 3) were the same as in Example 1.

4) 2-(5-Amino-1H-1,2,4-triazol-1-yl)-5-(5-(3,5-dichlorophenyl)-5-(trifluoromethyl)-4,5-dihydroisoxazol-3-yl)benzonitrile (Compound F, 0.3 g, 0.64 mmol) obtained in step 3), 4-chlorobenzoyl chloride (0.11 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,5-dichlorophenyl)-5-(trifluoromethyl)-4,5-dihydroisoxazol-3-yl)phenyl)-1H-1,2,4-triazol-5-yl)-4-chlorobenzamide as a light yellow solid, which was the phenyl isoxazole derivative containing a triazole ring of the present disclosure.

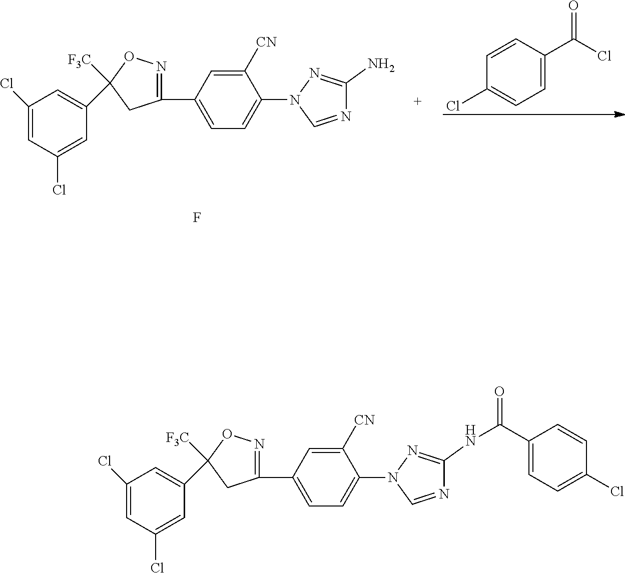

The yield of the phenyl isoxazole derivative in this example was determined to be 52%.

¹H NMR (500 MHz) δ 9.99 (s, 1H), 8.36 (s, 1H), 7.66 (d, J=1.6 Hz, 1H), 7.58 (dd, J=6.9, 1.5 Hz, 1H), 7.40 (dd, J=9.3, 4.1 Hz, 3H), 7.24 (t, J=1.4 Hz, 1H), 7.06 (dd, J=7.0, 1.3 Hz, 4H), 4.60 (d, J=14.7 Hz, 1H), 4.51 (d, J=14.8 Hz, 1H).

### Example 17

### Preparation of a Phenyl Isoxazole Derivative Containing a Triazole Ring

Steps 1) to 3) were the same as in Example 1.

2-(5-Amino-1H-1,2,4-triazol-1-yl)-5-(5-(3,5-dichlorophenyl)-5-(trifluoromethyl)-4,5-dihydroisoxazol-3-yl)benzonitrile (Compound F, 0.3 g, 0.64 mmol) obtained in step 3), 2-methylbenzoyl chloride (0.1 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,5-dichlorophenyl)-5-(trifluoromethyl)-4,5-dihydroisoxazol-3-yl)phenyl)-1H-1,2,4-triazol-5-yl)-2-methylbenzamide as a light yellow solid, which was the phenyl isoxazole derivative containing a triazole ring of the present disclosure.

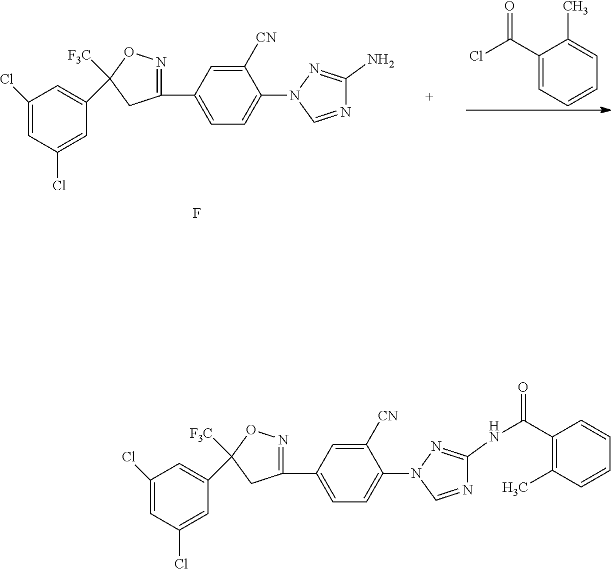

The yield of the phenyl isoxazole derivative in this example was determined to be 42%.

¹H NMR (600 MHz) δ 9.19 (s, 1H), 8.28 (s, 1H), 7.94 (d, J=6.9 Hz, 1H), 7.92 (d, J=1.4 Hz, 1H), 7.88 (dd, J=6.9, 1.6 Hz, 2H), 7.54 (d, J=5.9 Hz, 1H), 7.46 (s, 2H), 7.42 (dd, J=3.2, 1.8 Hz, 1H), 7.41-7.37 (m, 2H), 4.75 (s, J=13.7 Hz, 1H), 4.45 (d, J=13.7 Hz, 1H), 3.50 (s, 3H).

### Example 18

### Preparation of a Phenyl Isoxazole Derivative Containing a Triazole Ring

Steps 1) to 3) were the same as in Example 1.

4) 2-(5-Amino-1H-1,2,4-triazol-1-yl)-5-(5-(3,5-dichlorophenyl)-5-(trifluoromethyl)-4,5-dihydroisoxazol-3-yl)benzonitrile (Compound F, 0.3 g, 0.64 mmol) obtained in step 3), 3-methylbenzoyl chloride (0.1 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,5-dichlorophenyl)-5-(trifluoromethyl)-4,5-dihydroisoxazol-3-yl)phenyl)-1H-1,2,4-triazol-5-yl)-3-methylbenzamide as a light yellow solid, which was the phenyl isoxazole derivative containing a triazole ring of the present disclosure.

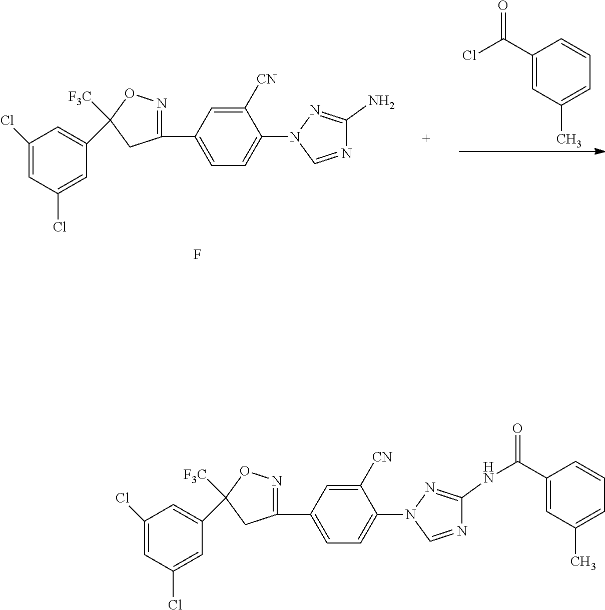

The yield of the phenyl isoxazole derivative in this example was determined to be 58.6%.

¹H NMR (600 MHz) δ 8.65 (s, 1H), 8.04 (s, 1H), 7.47 (dd, J=6.3, 4.2 Hz, 2H), 7.41 (dd, J=6.9, 1.3 Hz, 2H), 7.25 (s, 1H), 7.22 (dd, J=4.5, 2.3 Hz, 1H), 6.99 (d, J=0.8 Hz, 2H), 6.95 (t, J=1.3 Hz, 1H), 6.91 (d, J=3.9 Hz, 2H), 4.28 (d, J=13.7 Hz, 1H), 4.00 (d, J=13.7 Hz, 1H), 2.95 (s, 3H).

### Example 19

### Preparation of a Phenyl Isoxazole Derivative Containing a Triazole Ring

Steps 1) to 3) were the same as in Example 1.

4) 2-(5-Amino-1H-1,2,4-triazol-1-yl)-5-(5-(3,5-dichlorophenyl)-5-(trifluoromethyl)-4,5-dihydroisoxazol-3-yl)benzonitrile (Compound F, 0.3 g, 0.64 mmol) obtained in step 3), 4-methylbenzoyl chloride (0.1 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,5-dichlorophenyl)-5-(trifluoromethyl)-4,5-dihydroisoxazol-3-yl)phenyl)-1H-1,2,4-triazol-5-yl)-4-methylbenzamide as a light yellow solid, which was the phenyl isoxazole derivative containing a triazole ring of the present disclosure.

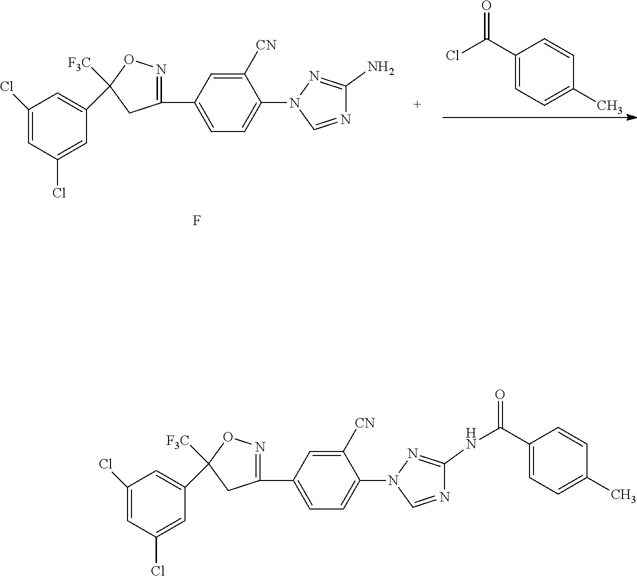

The yield of the phenyl isoxazole derivative in this example was determined to be 21.6%.

¹H NMR (600 MHz) δ 8.84 (s, 1H), 8.09 (s, 1H), 7.50 (d, J=7.0 Hz, 1H), 7.47 (d, J=1.5 Hz, 1H), 7.41 (dd, J=6.7, 1.5 Hz, 1H), 7.39 (d, J=6.5 Hz, 2H), 7.35 (d, J=6.4 Hz, 2H), 7.00 (d, J=0.8 Hz, 2H), 6.95 (t, J=1.3 Hz, 1H), 4.28 (d, J=13.7 Hz, 1H), 3.99 (d, J=13.7 Hz, 1H), 2.94 (s, 3H).

### Example 20

### Preparation of a Phenyl Isoxazole Derivative Containing a Triazole Ring

Step 1) was the same as in Example 1.

2) (E)-2-Fluoro-5-(hydroxyimino)methylbenzonitrile (Compound B, 16.4 g, 0.11 mol) obtained in step 1) and NCS (30 g, 0.22 mol) were added to a 250 mL round-bottom flask. The mixture was stirred at room temperature for 30 min. Subsequently, 1,2,3-trichloro-5-(3,3,3-trifluoroprop-1-en-2-yl)benzene (28.8 g, 0.12 mol) and triethylamine (13.3 g, 0.13 mol) were added. The mixture was stirred at room temperature for 18 h. After the reaction was completed, the mixture was washed with water, extracted with dichloromethane, dried over anhydrous sodium sulfate, concentrated under vacuum, and purified by column chromatography to obtain 5-(5-(3,4,5-trichlorophenyl)-5-(trifluoromethyl)-4,5-dihydroisoxazol-3-yl)-2-fluorobenzonitrile (Compound D) as a white solid.

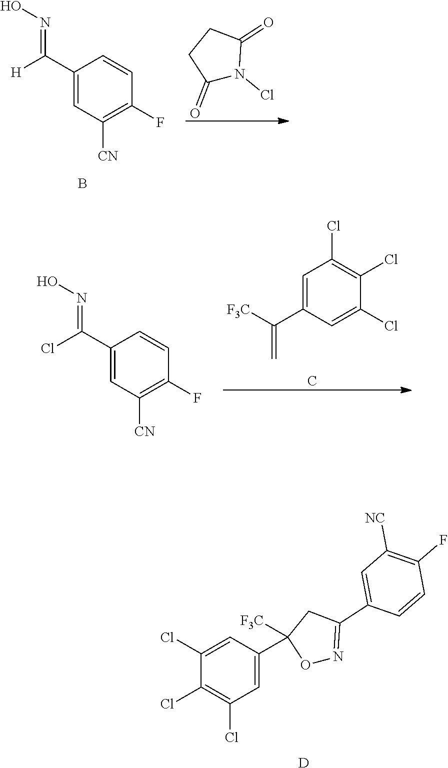

The yield of Compound D was determined to be 88.4%.

3) 3-Amino-1H-1,2,4-triazole (Compound E, 1.7 g, 20 mmol), potassium tert-butoxide (2.8 g, 2.5 mmol), and DMF (50 mL) were added to a 100 mL round-bottom flask. The mixture was stirred at room temperature for 1 h. Subsequently, 5-(5-(3,4,5-trichlorophenyl)-5-(trifluoromethyl)-4,5-dihydroisoxazol-3-yl)-2-fluorobenzonitrile (Compound D, 4 g, 10 mmol) obtained in step 2) was slowly added. The mixture was stirred and heated under reflux at 100° C. for 4 h. After the reaction was completed, the mixture was concentrated under vacuum and purified by column chromatography to obtain 2-(5-amino-1H-1,2,4-triazol-1-yl)-5-(5-(3,4,5-trichlorophenyl)-5-(trifluoromethyl)-4,5-dihydroisoxazol-3-yl)benzonitrile (Compound F) as a light yellow solid.

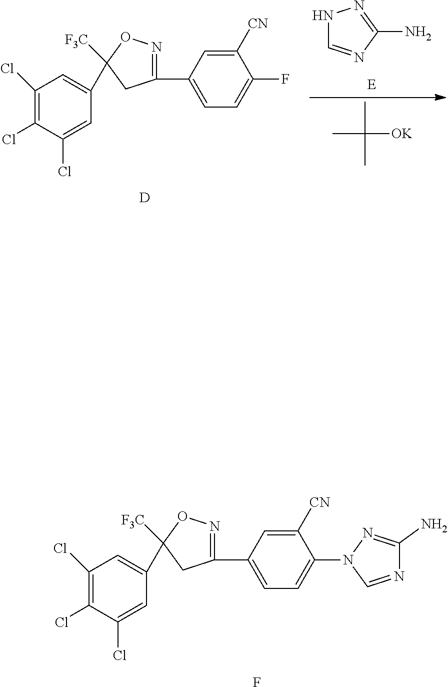

The yield of Compound F was determined to be 52%.

4) 2-(5-Amino-1H-1,2,4-triazol-1-yl)-5-(5-(3,4,5-trichlorophenyl)-5-(trifluoromethyl)-4,5-dihydroisoxazol-3-yl)benzonitrile (Compound F, 0.3 g, 0.64 mmol) obtained in step 3), 2-fluorobenzoyl chloride (0.09 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,4,5-trichlorophenyl)-5-(trifluoromethyl)-4,5-dihydroisoxazol-3-yl)phenyl)-1H-1,2,4-triazol-5-yl)-2-fluorobenzamide, which was the phenyl isoxazole derivative containing a triazole ring of the present disclosure, as a white solid.

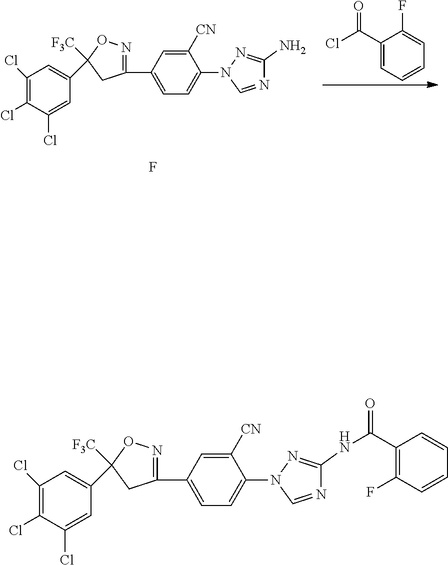

The yield of the phenyl isoxazole derivative in this example was determined to be 42%.

¹H NMR (600 MHz) δ 9.33 (d, J=13.8 Hz, 1H), 8.83 (s, 1H), 8.16 (td, J=7.8, 1.7 Hz, 1H), 8.09-8.08 (m, 1H), 8.07-8.02 (m, 2H), 7.64 (s, 2H), 7.59-7.54 (m, 1H), 7.35-7.30 (m, 1H), 7.21 (dd, J=11.8, 8.5 Hz, 1H), 4.13 (d, J=17.2 Hz, 1H), 3.78 (d, J=17.2 Hz, 1H).

### Example 21

### Preparation of a Phenyl Isoxazole Derivative Containing a Triazole Ring

Steps 1) to 3) were the same as in Example 20.

4) 2-(5-Amino-1H-1,2,4-triazol-1-yl)-5-(5-(3,4,5-trichlorophenyl)-5-(trifluoromethyl)-4,5-dihydroisoxazol-3-yl)benzonitrile (Compound F, 0.3 g, 0.64 mmol) obtained in step 3), 3-fluorobenzoyl chloride (0.09 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,4,5-trichlorophenyl)-5-(trifluoromethyl)-4,5-dihydroisoxazol-3-yl)phenyl)-1H-1,2,4-triazol-5-yl)-3-fluorobenzamide as a white solid, which was the phenyl isoxazole derivative containing a triazole ring of the present disclosure.

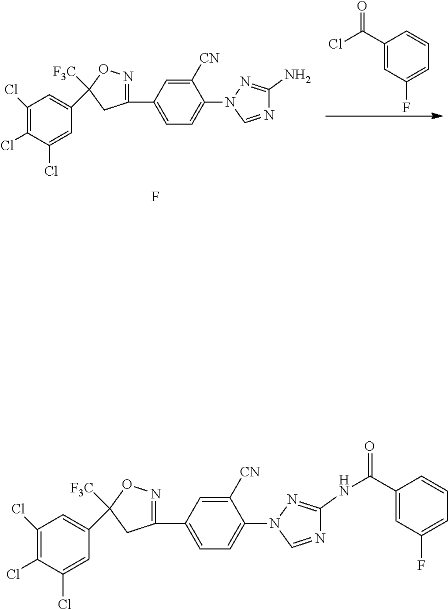

The yield of the phenyl isoxazole derivative in this example was determined to be 42%.

¹H NMR (600 MHz) δ 9.10 (s, 1H), 8.00 (s, 1H), 7.46 (d, J=1.2 Hz, 1H), 7.43-7.39 (m, 2H), 7.23 (d, J=6.2 Hz, 1H), 7.18-7.16 (m, 1H), 7.10 (s, 2H), 6.99 (td, J=6.4, 4.5 Hz, 1H), 6.82 (td, J=6.7, 1.8 Hz, 1H), 4.29 (d, J=13.8 Hz, 1H), 4.01 (d, J=13.8 Hz, 1H).

### Example 22

### Preparation of a Phenyl Isoxazole Derivative Containing a Triazole Ring

Steps 1) to 3) were the same as in Example 20.

4) 2-(5-Amino-1H-1,2,4-triazol-1-yl)-5-(5-(3,4,5-trichlorophenyl)-5-(trifluoromethyl)-4,5-dihydroisoxazol-3-yl)benzonitrile (Compound F, 0.3 g, 0.64 mmol) obtained in step 3), 4-fluorobenzoyl chloride (0.09 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,4,5-trichlorophenyl)-5-(trifluoromethyl)-4,5-dihydroisoxazol-3-yl)phenyl)-1H-1,2,4-triazol-5-yl)-4-fluorobenzamide as a white solid, which was the phenyl isoxazole derivative containing a triazole ring of the present disclosure.

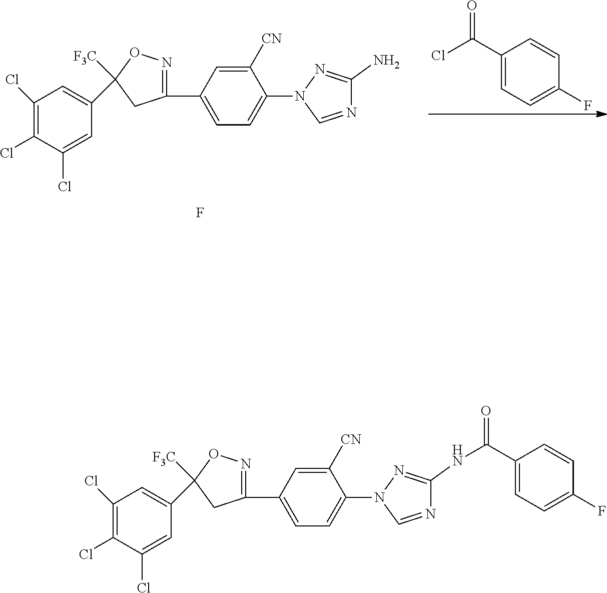

The yield of the phenyl isoxazole derivative in this example was determined to be 35.6%.

¹H NMR (600 MHz) δ 9.94 (s, 1H), 8.35 (s, 1H), 7.65 (d, J=1.5 Hz, 1H), 7.57 (dd, J=6.9, 1.5 Hz, 1H), 7.45-7.43 (m, 2H), 7.40 (d, J=6.9 Hz, 1H), 7.23 (s, 2H), 6.86 (t, J=7.0 Hz, 2H), 4.59 (d, J=14.7 Hz, 1H), 4.52 (d, J=14.7 Hz, 1H).

### Example 23

### Preparation of a Phenyl Isoxazole Derivative Containing a Triazole Ring

Steps 1) to 2) were the same as in Example 20.

3) 3-Nitro-1H-1,2,4-triazole (Compound E, 1.7 g, 20 mmol), potassium tert-butoxide (2.8 g, 2.5 mmol), and DMF (50 mL) were added to a 100 mL round-bottom flask. The mixture was stirred at room temperature for 1 h. Subsequently, 5-(5-(3,4,5-trichlorophenyl)-5-(trifluoromethyl)-4,5-dihydroisoxazol-3-yl)-2-fluorobenzonitrile (Compound D, 4 g, 10 mmol) obtained in step 2) was slowly added. The mixture was stirred and heated under reflux at 100° C. for 4 h. Finally, Fe powder (11 g, 20 mmol) was added, and the mixture was stirred and heated under reflux at 100° C. for 2 h. After the reaction was completed, the mixture was concentrated under vacuum and purified by column chromatography to obtain 2-(5-amino-1H-1,2,4-triazol-1-yl)-5-(5-(3,4,5-trichlorophenyl)-5-(trifluoromethyl)-4,5-dihydroisoxazol-3-yl)benzonitrile (Compound F) as a light yellow solid.

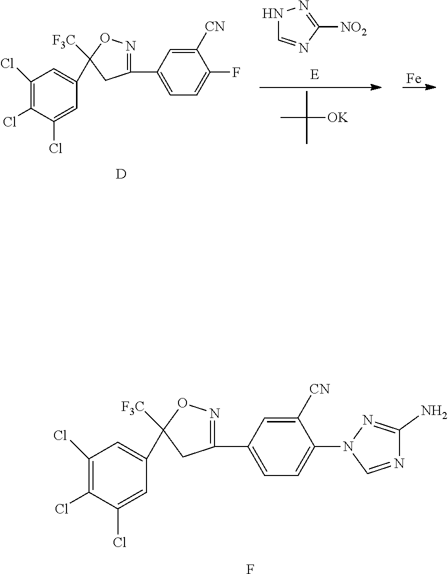

The yield of Compound F was determined to be 61%.

4) 2-(5-Amino-1H-1,2,4-triazol-1-yl)-5-(5-(3,4,5-trichlorophenyl)-5-(trifluoromethyl)-4,5-dihydroisoxazol-3-yl)benzonitrile (Compound F, 0.3 g, 0.64 mmol) obtained in step 3), 2-chlorobenzoyl chloride (0.09 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,4,5-trichlorophenyl)-5-(trifluoromethyl)-4,5-dihydroisoxazol-3-yl)phenyl)-1H-1,2,4-triazol-5-yl)-2-chlorobenzamide as a white solid, which was the phenyl isoxazole derivative containing a triazole ring of the present disclosure.

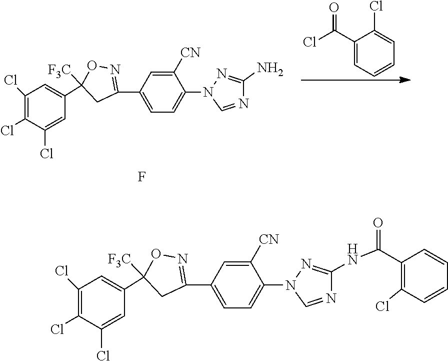

The yield of the phenyl isoxazole derivative in this example was determined to be 57%.

¹H NMR (500 MHz) δ 10.10 (s, 1H), 8.32 (s, 1H), 7.65 (d, J=1.4 Hz, 1H), 7.57 (dd, J=6.9, 1.4 Hz, 1H), 7.25 (d, J=15.2 Hz, 3H), 7.21-7.18 (m, 1H), 7.01-6.99 (m, 2H), 6.96-6.94 (m, 1H), 4.60 (d, J=14.7 Hz, 1H), 4.52 (d, J=14.8 Hz, 1H).

### Example 24

### Preparation of a Phenyl Isoxazole Derivative Containing a Triazole Ring

Steps 1) to 3) were the same as in Example 23.

4) 2-(5-Amino-1H-1,2,4-triazol-1-yl)-5-(5-(3,4,5-trichlorophenyl)-5-(trifluoromethyl)-4,5-dihydroisoxazol-3-yl)benzonitrile (Compound F, 0.3 g, 0.64 mmol) obtained in step 3), 3-chlorobenzoyl chloride (0.09 g, 0.64 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,4,5-trichlorophenyl)-5-(trifluoromethyl)-4,5-dihydroisoxazol-3-yl)phenyl)-1H-1,2,4-triazol-5-yl)-3-chlorobenzamide as a white solid, which was the phenyl isoxazole derivative containing a triazole ring of the present disclosure.

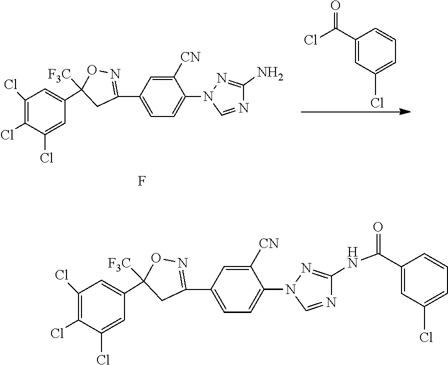

The yield of the phenyl isoxazole derivative in this example was determined to be 34.6%.

¹H NMR (500 MHz) δ 10.06 (s, 1H), 8.38 (s, 1H), 7.68 (d, J=1.5 Hz, 1H), 7.60 (dd, J=6.9, 1.7 Hz, 1H), 7.43 (t, J=4.3 Hz, 2H), 7.38-7.35 (m, 1H), 7.26 (s, 2H), 7.16-7.13 (m, 1H), 7.05 (t, J=6.3 Hz, 1H), 4.62 (d, J=14.7 Hz, 1H), 4.55 (d, J=14.8 Hz, 1H).

### Example 25

### Preparation of a Phenyl Isoxazole Derivative Containing a Triazole Ring

Steps 1) to 3) were the same as in Example 23.

4) 2-(5-Amino-1H-1,2,4-triazol-1-yl)-5-(5-(3,4,5-trichlorophenyl)-5-(trifluoromethyl)-4,5-dihydroisoxazol-3-yl)benzonitrile (Compound F, 0.3 g, 0.64 mmol) obtained in step 3), 4-chlorobenzoyl chloride (0.093 g, 0.6 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,4,5-trichlorophenyl)-5-(trifluoromethyl)-4,5-dihydroisoxazol-3-yl)phenyl)-1H-1,2,4-triazol-5-yl)-4-chlorobenzamide as a white solid, which was the phenyl isoxazole derivative containing a triazole ring of the present disclosure.

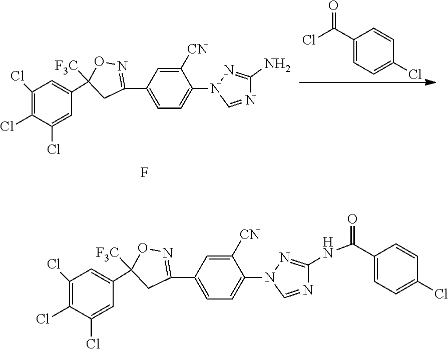

The yield of the phenyl isoxazole derivative in this example was determined to be 52%.

¹H NMR (500 MHz) δ 10.00 (s, 1H), 8.36 (s, 1H), 7.65 (d, J=1.6 Hz, 1H), 7.57 (dd, J=6.9, 1.6 Hz, 1H), 7.42-7.38 (m, 3H), 7.24 (s, 2H), 7.07-7.04 (m, 2H), 4.60 (d, J=14.7 Hz, 1H), 4.52 (d, J=14.8 Hz, 1H).

### Example 26

### Preparation of a Phenyl Isoxazole Derivative Containing a Triazole Ring

Steps 1) to 3) were the same as in Example 23.

4) 2-(5-Amino-1H-1,2,4-triazol-1-yl)-5-(5-(3,4,5-trichlorophenyl)-5-(trifluoromethyl)-4,5-dihydroisoxazol-3-yl)benzonitrile (Compound F, 0.3 g, 0.64 mmol) obtained in step 3), 2-methylbenzoyl chloride (0.093 g, 0.6 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,4,5-trichlorophenyl)-5-(trifluoromethyl)-4,5-dihydroisoxazol-3-yl)phenyl)-1H-1,2,4-triazol-5-yl)-2-methylbenzamide as a light yellow solid, which was the phenyl isoxazole derivative containing a triazole ring of the present disclosure.

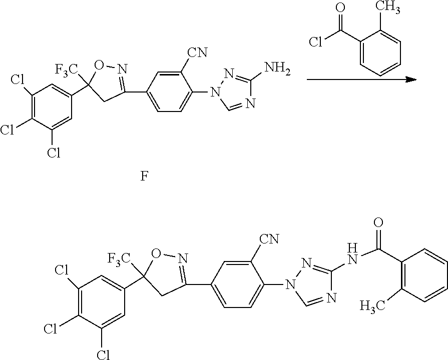

The yield of the phenyl isoxazole derivative in this example was determined to be 44.8%.

¹H NMR (400 MHz, CDCl₃) δ 10.40 (s, 1H), 8.11 (s, 1H), 8.09 (d, J=1.9 Hz, 1H), 8.06 (s, 1H), 8.01 (dd, J=8.7, 1.9 Hz, 1H), 7.65 (s, 2H), 7.60 (d, J=7.1 Hz, 1H), 7.47-7.40 (m, 1H), 7.34 (t, J=7.0 Hz, 2H), 4.13 (d, J=17.2 Hz, 1H), 3.76 (d, J=17.3 Hz, 1H), 2.54 (s, 3H).

### Example 27

### Preparation of a Phenyl Isoxazole Derivative Containing a Triazole Ring

Steps 1) to 3) were the same as in Example 23.

4) 2-(5-Amino-1H-1,2,4-triazol-1-yl)-5-(5-(3,4,5-trichlorophenyl)-5-(trifluoromethyl)-4,5-dihydroisoxazol-3-yl)benzonitrile (Compound F, 0.3 g, 0.64 mmol) obtained in step 3), 3-methylbenzoyl chloride (0.093 g, 0.6 mmol), triethylamine (0.1 g, 0.96 mmol), and dichloroethane (20 mL) were added to a 50 mL round-bottom flask. The mixture was stirred and heated under reflux at 120° C. for 4 h. After the reaction was completed, the mixture was concentrated under vacuum and purified by column chromatography to obtain N-(1-(2-cyano-4-(5-(3,4,5-trichlorophenyl)-5-(trifluoromethyl)-4,5-dihydroisoxazol-3-yl)phenyl)-1H-1,2,4-triazol-5-yl)-3-methylbenzamide as a light yellow solid, which was the phenyl isoxazole derivative containing a triazole ring of the present disclosure.

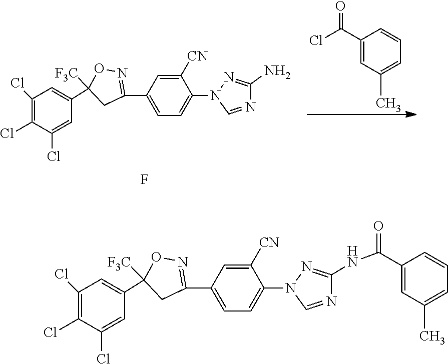

The yield of the phenyl isoxazole derivative in this example was determined to be 51.5%.

¹H NMR (400 MHz, CDCl₃) δ 9.62 (s, 1H), 8.70 (s, 1H), 8.09 (d, J=1.7 Hz, 1H), 8.03 (dt, J=8.7, 5.2 Hz, 2H), 7.76 (dd, J=9.0, 4.7 Hz, 2H), 7.64 (s, 2H), 7.40 (d, J=4.7 Hz, 2H), 4.14 (d, J=17.3 Hz, 1H), 3.79 (d, J=17.3 Hz, 1H), 2.43 (s, 3H).

### Efficacy Tests

1. Insecticidal Activity Test Against Plutella xylostella

Fresh cabbage leaves were soaked in a solution of the compound (i.e., the phenyl isoxazole derivatives prepared in Examples 1-27) at a concentration of 50 μg/mL for 30 seconds, air-dried, and placed in a culture dish lined with filter paper. Fifteen third-instar Plutella xylostella larvae were then transferred into the culture dish and cultured in an incubator at 26° C. and 85% relative humidity under a 16/8-h light/dark cycle for 2 days. After the cultivation was completed, a mortality rate of Plutella xylostella was evaluated (larvae that could not crawl normally upon gentle touch were considered dead). The test was performed in triplicate.

$$Mortality rate = ( X - Y ) / X \times 100 \% ;$$

wherein X is the number of live insects before cultivation, and Y is the number of live insects after cultivation.

The insecticidal activity test results of the phenyl isoxazole derivatives prepared in Examples 1-27 against Plutella xylostella are shown in Table 1.

TABLE 1
Insecticidal Activity Test Results of Phenyl Isoxazole
Derivatives Against Plutella xylostella

| | Mortality Rate (%) | | Mortality Rate (%) |
|---|---|---|---|
| Example 1 | 100 | Example 2 | 100 |
| Example 3 | 100 | Example 4 | 100 |
| Example 5 | 100 | Example 6 | 100 |
| Example 7 | 100 | Example 8 | 100 |
| Example 9 | 100 | Example 10 | 100 |
| Example 11 | 100 | Example 12 | 100 |
| Example 13 | 100 | Example 14 | 100 |
| Example 15 | 100 | Example 16 | 100 |
| Example 17 | 100 | Example 18 | 100 |
| Example 19 | 100 | Example 20 | 100 |
| Example 21 | 100 | Example 22 | 100 |
| Example 23 | 100 | Example 24 | 100 |
| Example 25 | 100 | Example 26 | 100 |
| Example 27 | 100 | Fluxametamide | 100 |

The data in Table 1 showed that the phenyl isoxazole derivatives provided by the present disclosure achieve a mortality rate of up to 100% against Plutella xylostella at a concentration of 50 μg/mL, which is comparable to fluxametamide. This indicates that the phenyl isoxazole derivatives provided by the present disclosure possess insecticidal activity against Plutella xylostella and can be used as active pesticide ingredients for controlling agricultural pests.

2. Insecticidal Activity Test Against Spodoptera frugiperda

A solution of the compound (i.e., the phenyl isoxazole derivatives prepared in Examples 1-27) at a concentration of 50 μg/mL was added to a 24-well plate, and 24 third-instar Spodoptera frugiperda larvae were placed in each well. The plate was incubated in an incubator at 26° C. and 85% relative humidity under a 16/8-h light/dark cycle for 2 days. After the cultivation was completed, the mortality rate of Spodoptera frugiperda was evaluated (larvae that could not crawl normally upon gentle touch were considered dead). The test was performed in triplicate.

$$Mortality rate = ( X - Y ) / X \times 100 \% ;$$

wherein X is the number of live insects before cultivation, and Y is the number of live insects after cultivation.

The insecticidal activity test results of the phenyl isoxazole derivatives prepared in Examples 1-27 against Spodoptera frugiperda are shown in Table 2.

TABLE 2
Insecticidal Activity Test Results of Phenyl Isoxazole
Derivatives Against Spodoptera frugiperda

| | Mortality Rate (%) | | Mortality Rate (%) |
|---|---|---|---|
| Example 1 | 100 | Example 2 | 100 |
| Example 3 | 100 | Example 4 | 100 |
| Example 5 | 100 | Example 6 | 100 |
| Example 7 | 100 | Example 8 | 100 |
| Example 9 | 100 | Example 10 | 100 |
| Example 11 | 100 | Example 12 | 100 |
| Example 13 | 100 | Example 14 | 100 |
| Example 15 | 100 | Example 16 | 100 |
| Example 17 | 100 | Example 18 | 100 |
| Example 19 | 100 | Example 20 | 100 |
| Example 21 | 100 | Example 22 | 100 |
| Example 23 | 100 | Example 24 | 100 |
| Example 25 | 100 | Example 26 | 100 |
| Example 27 | 100 | Fluxametamide | 100 |

The data in Table 2 showed that the phenyl isoxazole derivatives provided by the present disclosure achieve a mortality rate of up to 100% against Spodoptera frugiperda at a concentration of 50 μg/mL, which is comparable to fluxametamide. This indicates that the phenyl isoxazole derivatives provided by the present disclosure possess insecticidal activity against Spodoptera frugiperda and can be used as active pesticide ingredients for controlling agricultural pests.

3. Insecticidal Activity Test Against Helicoverpa armigera

A solution of the compound (i.e., the phenyl isoxazole derivatives prepared in Examples 1-27) at a concentration of 50 μg/mL was added to a 24-well plate, and 24 third-instar Helicoverpa armigera larvae were placed in each well. The plate was incubated in an incubator at 26° C. and 85% relative humidity under a 16/8-h light/dark cycle for 2 days. After the cultivation was completed, the mortality rate of Helicoverpa armigera was evaluated (larvae that could not crawl normally upon gentle touch were considered dead). The test was performed in triplicate.

$$Mortality rate = ( X - Y ) / X \times 100 \% ;$$

wherein X is the number of live insects before cultivation, and Y is the number of live insects after cultivation.

The insecticidal activity test results of the phenyl isoxazole derivatives prepared in Examples 1-27 against Helicoverpa armigera are shown in Table 3.

TABLE 3
Insecticidal Activity Test Results of Phenyl Isoxazole
Derivatives Against Helicoverpa armigera

| | Mortality Rate (%) | | Mortality Rate (%) |
|---|---|---|---|
| Example 1 | 100 | Example 2 | 100 |
| Example 3 | 100 | Example 4 | 100 |
| Example 5 | 100 | Example 6 | 100 |
| Example 7 | 100 | Example 8 | 100 |
| Example 9 | 100 | Example 10 | 100 |
| Example 11 | 100 | Example 12 | 100 |
| Example 13 | 100 | Example 14 | 100 |
| Example 15 | 100 | Example 16 | 100 |
| Example 17 | 100 | Example 18 | 100 |
| Example 19 | 100 | Example 20 | 100 |
| Example 21 | 100 | Example 22 | 100 |
| Example 23 | 100 | Example 24 | 100 |
| Example 25 | 100 | Example 26 | 100 |
| Example 27 | 100 | Fluxametamide | 100 |

The data in Table 3 showed that the phenyl isoxazole derivatives provided by the present disclosure achieve a mortality rate of up to 100% against Helicoverpa armigera at a concentration of 50 μg/mL, which is comparable to fluxametamide. This indicates that the phenyl isoxazole derivatives provided by the present disclosure possess insecticidal activity against Helicoverpa armigera and can be used as active pesticide ingredients for controlling agricultural pests.

4. Insecticidal Activity Test Against Ostrinia nubilalis

A solution of the compound (i.e., the phenyl isoxazole derivatives prepared in Examples 1-27) at a concentration of 50 μg/mL was added to a 24-well plate, and 24 third-instar Ostrinia nubilalis larvae were placed in each well. The plate was incubated in an incubator at 26° C. and 85% relative humidity under a 16/8-h light/dark cycle for 2 days. After the cultivation was completed, the mortality rate of Ostrinia nubilalis was evaluated (larvae that could not crawl normally upon gentle touch were considered dead). The test was performed in triplicate.

$$Mortality rate = ( X - Y ) / X \times 100 \% ;$$

wherein X is the number of live insects before cultivation, and Y is the number of live insects after cultivation.

The insecticidal activity test results of the phenyl isoxazole derivatives prepared in Examples 1-27 against Ostrinia nubilalis are shown in Table 4.

TABLE 4
Insecticidal Activity Test Results of Phenyl Isoxazole
Derivatives Against Ostrinia nubilalis

| | Mortality Rate (%) | | Mortality Rate (%) |
|---|---|---|---|
| Example 1 | 100 | Example 2 | 100 |
| Example 3 | 100 | Example 4 | 100 |
| Example 5 | 100 | Example 6 | 100 |
| Example 7 | 100 | Example 8 | 100 |
| Example 9 | 100 | Example 10 | 100 |
| Example 11 | 100 | Example 12 | 100 |
| Example 13 | 100 | Example 14 | 100 |
| Example 15 | 100 | Example 16 | 100 |
| Example 17 | 100 | Example 18 | 100 |
| Example 19 | 100 | Example 20 | 100 |
| Example 21 | 100 | Example 22 | 100 |
| Example 23 | 100 | Example 24 | 100 |
| Example 25 | 100 | Example 26 | 100 |
| Example 27 | 100 | Fluxametamide | 100 |

The data in Table 4 showed that the phenyl isoxazole derivatives provided by the present disclosure achieve a mortality rate of up to 100% against Ostrinia nubilalis at a concentration of 50 μg/mL, which is comparable to fluxametamide. This indicates that the phenyl isoxazole derivatives provided by the present disclosure possess insecticidal activity against Ostrinia nubilalis and can be used as active pesticide ingredients for controlling agricultural pests.

### 5. Insecticidal Activity Test Against Aphids

Broad bean seedlings (3-5 cm) were rinsed with clean water, and transplanted into plastic culture dishes. An appropriate amount of quartz sand was added to the plastic culture dishes, and 4 mL of water was then added dropwise for moisture retention.

Thirty to fifty aphids were picked from broad bean plants and transferred onto the broad bean seedlings using a soft brush. The seedlings were covered with a nylon mesh. After all aphids had climbed onto the bean seedlings, a solution of the compound (i.e., the phenyl isoxazole derivatives prepared in Examples 1-27) at a concentration of 50 μg/mL was sprayed onto the seedlings using a small sprayer. The dishes were placed in an incubator at 26° C. and 70% relative humidity under a 14/10-h light/dark cycle for 2 days. After the cultivation was completed, the mortality rate of aphids was evaluated (larvae that could not crawl normally upon gentle touch were considered dead). The test was performed in triplicate.

$$Mortality rate = ( X - Y ) / X \times 100 \% ;$$

wherein X is the number of live insects before cultivation, and Y is the number of live insects after cultivation.

The insecticidal activity test results of the phenyl isoxazole derivatives prepared in Examples 1-27 against aphids are shown in Table 5.

TABLE 5
Insecticidal Activity Test Results of Phenyl
Isoxazole Derivatives Against Aphids

| | Mortality Rate (%) | | Mortality Rate (%) |
|---|---|---|---|
| Example 1 | 100 | Example 2 | 100 |
| Example 3 | 100 | Example 4 | 100 |
| Example 5 | 100 | Example 6 | 100 |
| Example 7 | 100 | Example 8 | 100 |
| Example 9 | 100 | Example 10 | 100 |
| Example 11 | 100 | Example 12 | 100 |
| Example 13 | 100 | Example 14 | 100 |
| Example 15 | 100 | Example 16 | 100 |
| Example 17 | 100 | Example 18 | 100 |
| Example 19 | 100 | Example 20 | 100 |
| Example 21 | 100 | Example 22 | 100 |
| Example 23 | 100 | Example 24 | 100 |
| Example 25 | 100 | Example 26 | 100 |
| Example 27 | 100 | Fluxametamide | 100 |

The data in Table 5 showed that the phenyl isoxazole derivatives provided by the present disclosure achieve a mortality rate of up to 100% against aphids at a concentration of 50 μg/mL, which is comparable to fluxametamide. This indicates that the phenyl isoxazole derivatives provided by the present disclosure possess insecticidal activity against aphids and can be used as active pesticide ingredients for controlling agricultural pests.

### 6. Toxicity Test

The compound (i.e., the phenyl isoxazole derivatives prepared in Examples 1-27) was dissolved in dimethyl sulfoxide and then diluted with sugar water (1 g:1 g) to a concentration of 50 μg/mL. The resulting solution of the compound was added to a feeding trough, and 15 honeybees were placed in a bee rearing box. The honeybees were allowed to be fed for 5 hours, and then the feeding trough was removed. The bee rearing box was then placed in an incubator at 26° C. and 70% relative humidity under a 14/10-h light/dark cycle for 2 days. After the cultivation was completed, bee mortality was evaluated (larvae that could not crawl normally upon gentle touch were considered dead). The test was performed in triplicate.

$$Mortality rate = ( X - Y ) / X \times 100 \% ;$$

wherein X is the number of live insects before cultivation, and Y is the number of live insects after cultivation.

The toxicity test results of the phenyl isoxazole derivatives prepared in Examples 1-27 are shown in Table 6.

TABLE 6
Toxicity Test Results of Phenyl Isoxazole Derivatives

| | Mortality Rate (%) | | Mortality Rate (%) |
|---|---|---|---|
| Example 1 | 0 | Example 2 | 0 |
| Example 3 | 0 | Example 4 | 0 |
| Example 5 | 0 | Example 6 | 0 |
| Example 7 | 0 | Example 8 | 0 |
| Example 9 | 0 | Example 10 | 0 |
| Example 11 | 0 | Example 12 | 0 |
| Example 13 | 0 | Example 14 | 0 |
| Example 15 | 0 | Example 16 | 0 |
| Example 17 | 0 | Example 18 | 0 |
| Example 19 | 0 | Example 20 | 0 |
| Example 21 | 0 | Example 22 | 0 |
| Example 23 | 0 | Example 24 | 0 |
| Example 25 | 0 | Example 26 | 0 |
| Example 27 | 0 | Fluxametamide | 100 |

The data in Table 6 showed that the phenyl isoxazole provided by the present disclosure exhibited a 0% mortality rate against honeybees at a concentration of 50 μg/mL, whereas fluxametamide exhibited a 100% mortality rate against honeybees. This indicates that the phenyl isoxazole derivatives containing a triazole ring provided by the present disclosure have low toxicity and can be used as active pesticide ingredients for controlling agricultural pests.

Finally, it should be noted that the above embodiments are merely intended to illustrate the technical solutions of the present disclosure and not to limit them. Although the present disclosure has been described in detail with reference to the foregoing embodiments, those of ordinary skill in the art should understand that modifications or equivalent substitutions can still be made to the specific embodiments of the present disclosure, and any such modifications or equivalent substitutions that do not depart from the spirit and scope of the present disclosure shall be covered by the scope of the appended claims.

What is claimed is:

1. A phenyl isoxazole derivative containing a triazole ring, having a structural formula of: 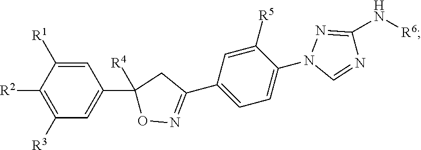
wherein, R¹, R², and R³ are each independently selected from the group consisting of hydrogen, halogen, cyano, nitro, C₁-C₆ alkyl, C₁-C₆ haloalkyl, and C₁-C₆ haloalkoxy;
R⁴ is selected from the group consisting of hydrogen, halogen, cyano, nitro, C₁-C₆ alkyl, and C₁-C₆ haloalkyl;
R⁵ is selected from the group consisting of hydrogen, halogen, cyano, nitro, hydroxyl, amino, mercapto, C₁-C₆ alkoxy, C₁-C₆ alkenyl, and C₁-C₆ cycloalkyl; and
R⁶ is selected from the group consisting of trifluoroacetyl, dichloroacetyl, trichloroacetyl, methyl alkoxyoxalyl, benzoyl, 2-chloro-4-fluorobenzoyl, 3-chloro-3-chlorobenzoyl, 2-trifluoromethylbenzoyl, 3-trifluoromethylbenzoyl, 4-trifluoromethylbenzoyl, 2-fluorobenzoyl, 3-fluorobenzoyl, 4-fluorobenzoyl, 2-chlorobenzoyl, 3-chlorobenzoyl, 4-chlorobenzoyl, 2-methylbenzoyl, 3-methylbenzoyl, and 4-methylbenzoyl.

2. A method for preparing the phenyl isoxazole derivative containing a triazole ring according to claim 1, comprising the following steps:
1) mixing Compound A, hydroxylamine hydrochloride, sodium carbonate, and an organic solvent for reaction to obtain Compound B, wherein Compound A has a structural formula of 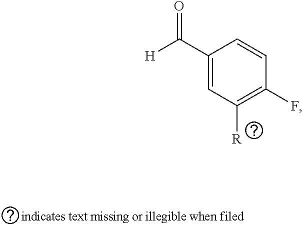
and Compound B has a structural formula of 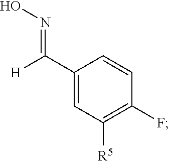
2) subjecting Compound B obtained in step 1) to a chlorination reaction to obtain a chlorinated product, and mixing the chlorinated product, Compound C, and an organic solvent for reaction to obtain Compound D, wherein Compound C has a structural formula of 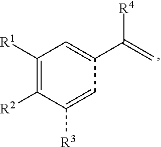
and Compound D has a structural formula of 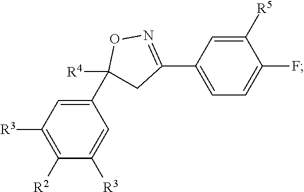
3) mixing Compound D obtained in step 2), Compound E, a base, and an organic solvent for reaction to obtain Compound F, wherein Compound E has a structural formula of 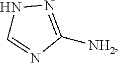
and Compound F has a structural formula of 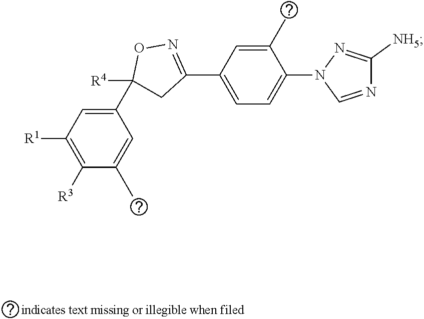
and
4) mixing Compound F obtained in step 3), a reactant containing a R⁶ group, and a base for reaction to obtain the phenyl isoxazole derivative containing a triazole ring.

3. The method according to claim 2, wherein in step 1), the organic solvent is one or more selected from the group consisting of methanol, ethanol, triethylamine, dichloromethane, chloroform, ethyl acetate, acetonitrile, toluene, xylene, tetrahydrofuran, N,N-dimethylformamide, n-propanol, and water; and a molar ratio of compound A, hydroxylamine hydrochloride, sodium carbonate, and the organic solvent is 1:(1-3):(1-5):(2-5).

4. The method according to claim 2, wherein in step 2), a chlorinating agent for the chlorination reaction is N-chlorosuccinimide; the organic solvent is one or more selected from the group consisting of methanol, ethanol, triethylamine, dichloromethane, chloroform, ethyl acetate, acetonitrile, toluene, xylene, tetrahydrofuran, N,N-dimethylformamide, n-propanol, and water; and a molar ratio of compound B, the chlorinating agent, compound C, and the organic solvent is 1:(1-4):(1-3):(2-8).

5. The method according to claim 2, wherein in step 3), the base is lithium hydroxide, sodium hydroxide, potassium hydroxide, calcium hydroxide, potassium tert-butoxide, aqueous ammonia, methylamine, ethylamine, propylamine, dimethylamine, diethylamine, dipropylamine, trimethylamine, triethylamine, tripropylamine, N,N-diisopropylethylamine, sodium acetate, potassium acetate, or sodium bicarbonate; the organic solvent is one or more selected from the group consisting of methanol, ethanol, triethylamine, dichloromethane, chloroform, ethyl acetate, acetonitrile, toluene, xylene, tetrahydrofuran, N,N-dimethylformamide, n-propanol, and water; and a molar ratio of compound D, compound E, the base, and the organic solvent is 1:(1-4):(1-3):(2-8).

6. The method according to claim 2, wherein in step 4), the base is lithium hydroxide, sodium hydroxide, potassium hydroxide, calcium hydroxide, potassium tert-butoxide, aqueous ammonia, methylamine, ethylamine, propylamine, dimethylamine, diethylamine, dipropylamine, trimethylamine, triethylamine, tripropylamine, N,N-diisopropylethylamine, sodium acetate, potassium acetate, or sodium bicarbonate; and a molar ratio of compound F, the reactant containing the R⁶ group, and the base is 1:(1-4):(1-3).

7. A phenyl isoxazole derivative containing a triazole ring, selected from the group consisting of:

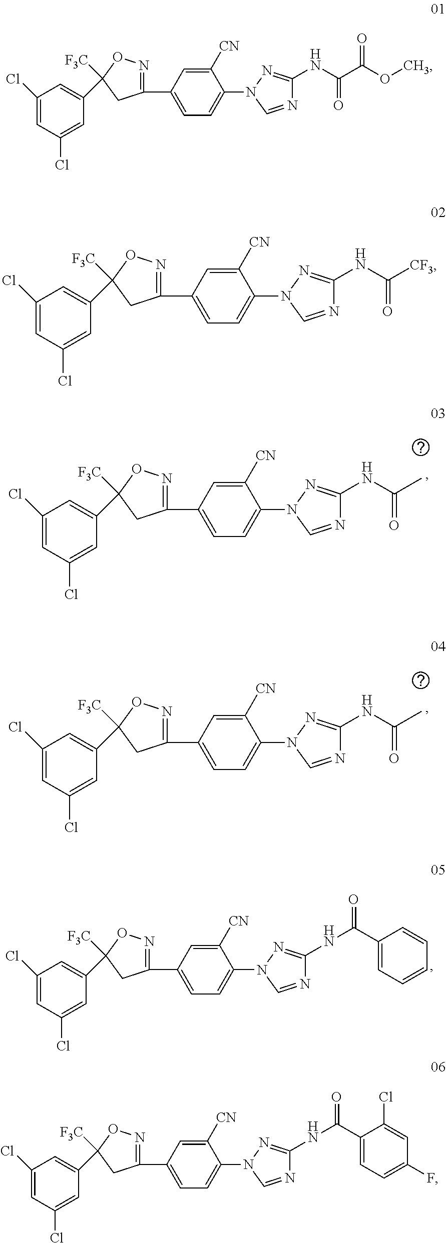

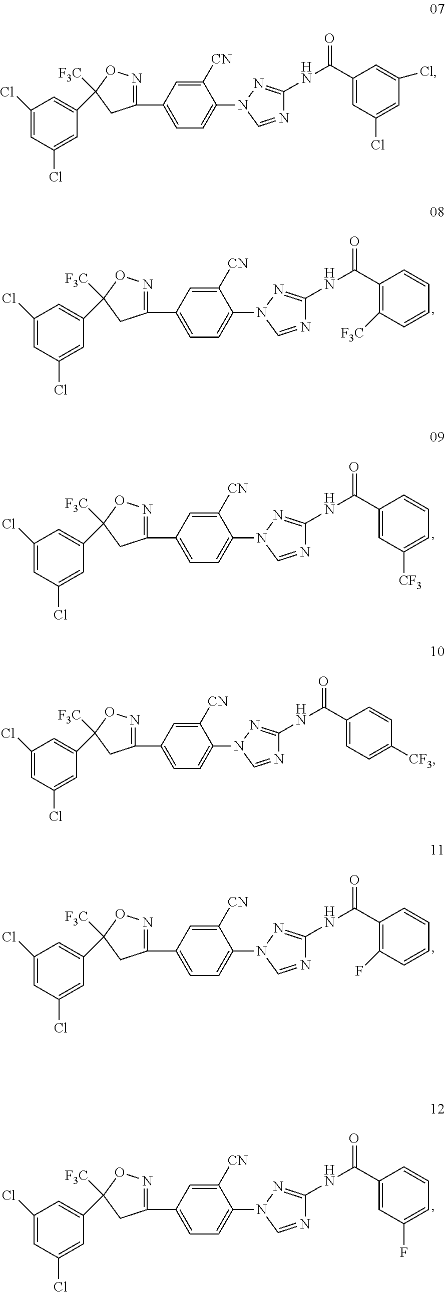

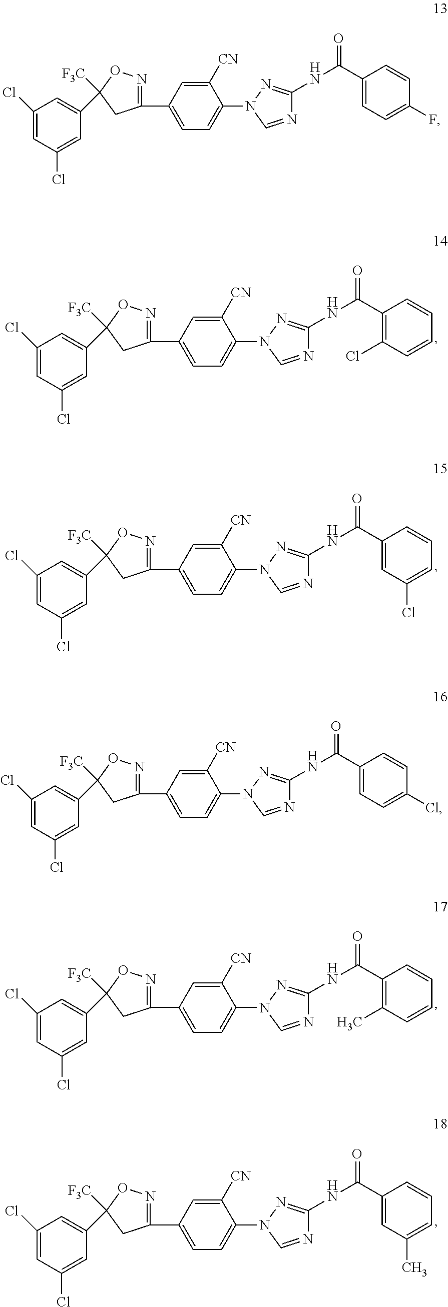

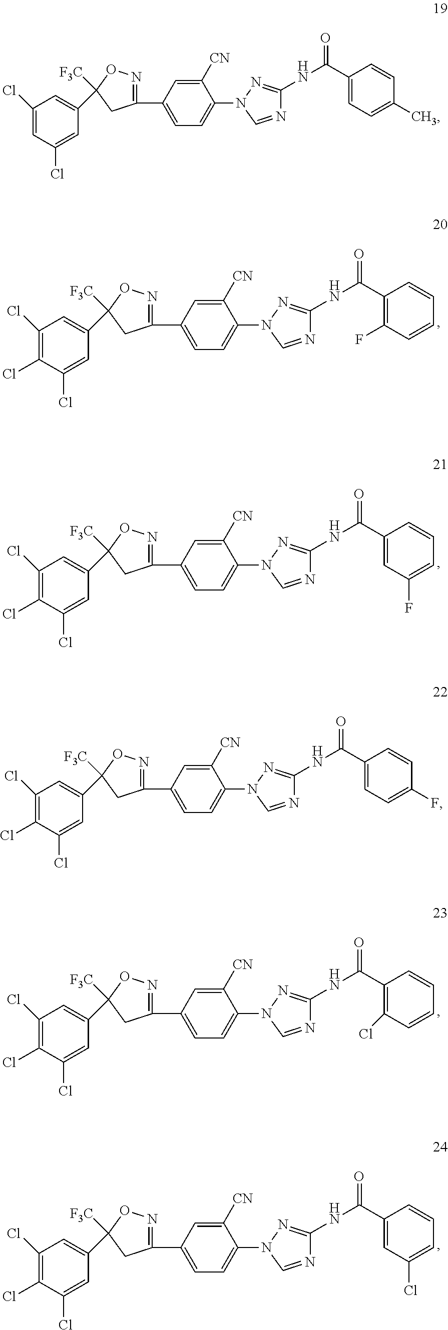

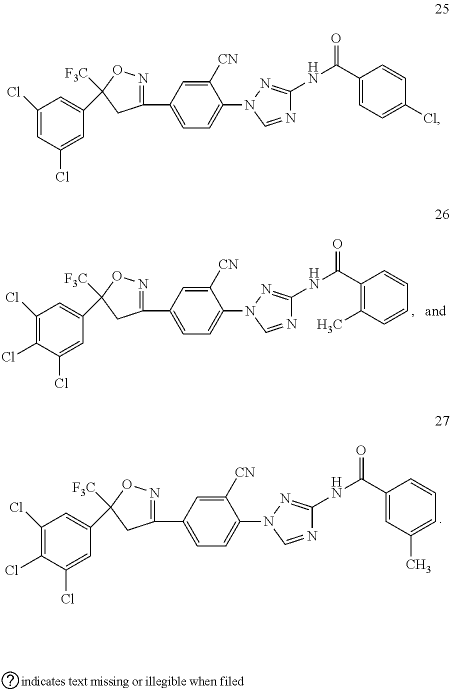 [SMILES: N#Cc1cc(C2=NOC(c3cc(Cl)cc(Cl)c3)(C(F)(F)F)C2)ccc1-n1cnc(NC(=O)c2ccc(Cl)cc2)n1 ; Cc1ccccc1C(=O)Nc1ncn(-c2ccc(C3=NOC(c4cc(Cl)c(Cl)c(Cl)c4)(C(F)(F)F)C3)cc2C#N)n1 ; "," ; "and" ; Cc1cccc(C(=O)Nc2ncn(-c3ccc(C4=NOC(c5cc(Cl)c(Cl)c(Cl)c5)(C(F)(F)F)C4)cc3C#N)n2)c1 ; "."]

8. An application of the phenyl isoxazole derivative containing a triazole ring according to claim 1 as an active pesticide ingredient in the control of agricultural pests, wherein the agricultural pests are selected from the group consisting of Plutella xylostella, Spodoptera frugiperda, Helicoverpa armigera, Ostrinia nubilalis, and aphids.
